(19) United States
(12) Patent Application Publication
(10) Pub. No.: US 2023/0271901 A1
(43) Pub. Date: Aug. 31, 2023

(54) METHOD FOR PREPARING DEUTERATED AROMATIC COMPOUND AND DEUTERATED REACTION COMPOSITION

(71) Applicant: LG CHEM, LTD., Seoul (KR)

(72) Inventors: Dai Seung CHOI, Daejeon (KR); Seungyeon HWANG, Daejeon (KR); Dongmin JEONG, Daejeon (KR); Bethy KIM, Daejeon (KR)

(21) Appl. No.: 18/017,716

(22) PCT Filed: Aug. 27, 2021
(86) PCT No.: PCT/KR2021/011539

§ 371 Date: Jan. 24, 2023

(30) Foreign Application Priority Data
Aug. 27, 2020 (KR) 10-2020-0108192
Dec. 18, 2020 (KR) 10-2020-0178795

(51) Int. Cl. C07C 15/28; C07B 59/00; C07C 15/24; C07D 307/91; C07C 17/395; C07C 37/86; H10K 85/60

(52) U.S. Cl. CPC C07C 15/28; C07B 59/001; C07B 59/002; C07C 15/24; C07D 307/91; C07C 17/395; C07C 37/86; H10K 85/626; H10K 85/6574; C07B 2200/05; C07C 2602/10; C07C 2603/24; C07C 2603/42

(57) ABSTRACT

Provided is a method for producing a deuterated aromatic compound and a deuterated reaction composition. The method includes performing a deuterated reaction of an aromatic compound including one or more hydrocarbon aromatic rings using a solution comprising heavy water, an organic compound which can be hydrolyzed by the heavy water, the aromatic compound, and an organic solvent. The method has an advantage in that impurities due to hydrogen gas are not generated, the deuterium substitution rate is high, and the deuterated reaction can be performed under a lower pressure and a lower temperature.

### CROSS-REFERENCE TO RELATED APPLICATION(S)

This application is a National Stage Application of International Application No. PCT/KR2021/011539 filed on Aug. 27, 2021, which claims priority to and the benefit of Korean Patent Application Nos. 10-2020-0108192 and 10-2020-0178795 filed in the Korean Intellectual Property Office on Aug. 27, 2020 and Dec. 18, 2020, respectively, the entire contents of which are incorporated herein by reference.

### TECHNICAL FIELD

The present specification relates to a method for producing a deuterated aromatic compound and a deuterated reaction composition.

### BACKGROUND

Compounds including deuterium are used for various purposes. For example, compounds including deuterium can be frequently used not only as labeling compounds for elucidating the mechanism of a chemical reaction or elucidating a material metabolism, but also for drugs, pesticides, organic EL materials and other purposes.

A method of deuterium substitution of an aromatic compound is known in order to improve the lifespan of an organic light emitting device (OLED) material. The principle of such an effect is that while the LUMO energy of C-D bond is lower than that of C—H bond during deuterium substitution, the life characteristics of the OLED material are improved.

When a deuterated reaction was performed on one or more aromatic compounds using an existing heterogeneous catalytic reaction, there was a problem in that by-products due to side reactions continued to be generated. The by-products are caused by a hydrogenation reaction generated by hydrogen gas, and in order to remove the by-products, attempts have also been made to increase the purity through the purification process after the reaction, but it was difficult to obtain high purity because there was no difference in melting point and solubility from existing materials. When the reaction is performed without hydrogen gas in order to alleviate the problem, the reaction needs to be performed at a very high temperature (about 220° C. or higher), which can pose a safety problem in the process.

### BRIEF DESCRIPTION

### Technical Problem

The present specification has been made in an effort to provide a method for producing a deuterated aromatic compound and a deuterated reaction composition.

### Technical Solution

The present specification provides a method for producing a deuterated aromatic compound, the method including: performing a deuterated reaction of an aromatic compound including one or more hydrocarbon aromatic rings using a solution including heavy water, an organic compound which can be hydrolyzed by the heavy water, the aromatic compound, and an organic solvent.

In the method for producing a deuterated aromatic compound of the present specification, the performing of the deuterated reaction of the aromatic compound includes:

preparing a solution including an aromatic compound including one or more hydrocarbon aromatic rings, heavy water, an organic compound which can be hydrolyzed by the heavy water, and an organic solvent; and performing a deuterated reaction of the aromatic compound by heating the solution for a deuterated reaction.

In the method for producing a deuterated aromatic compound of the present specification, the organic compound which can be hydrolyzed by the heavy water includes at least one compound of the following Chemical Formulae 1 to 4:

R1-C(O)OC(O)—R2 [Chemical Formula 1]

R3-S(O₂)OS(O₂)—R4 [Chemical Formula 2]

R5-C(O)O—R6 [Chemical Formula 3]

R7-CONH—R8 [Chemical Formula 4]

wherein in Chemical Formulae 1 to 4, R1 to R8 are the same as or different from each other, and are each independently a monovalent organic group which is unsubstituted or substituted with a halogen group.

In the method for producing a deuterated aromatic compound of the present specification, the organic compound which can be hydrolyzed by the heavy water includes at least one of trifluoromethanesulfonic anhydride, trifluoroacetic anhydride, acetic anhydride, methanesulfonic anhydride, methyl acetate, ethyl acetate and dimethylacetamide.

Further, the present specification provides a deuterated reaction composition including an aromatic compound including one or more hydrocarbon aromatic rings, heavy water, an organic compound which can be hydrolyzed by the heavy water, and an organic solvent.

In the method for producing a deuterated aromatic compound or deuterated reaction composition of the present specification, the organic solvent is selected from the group consisting of a hydrocarbon chain which is unsubstituted or substituted with a group selected from an alkyl group and a halogen group; an aliphatic hydrocarbon ring which is unsubstituted or substituted with a group selected from an alkyl group and a halogen group; an aromatic hydrocarbon ring which is unsubstituted or substituted with a group selected from an alkyl group and a halogen group; an alkene compound which is unsubstituted or substituted with a group selected from an alkyl group and a halogen group; a straight-chained or branched heterochain; a substituted or unsubstituted aliphatic hetero ring; and a substituted or unsubstituted aromatic hetero ring.

In the method for producing a deuterated aromatic compound or deuterated reaction composition of the present specification, the organic solvent is selected from the group consisting of cyclohexane, methylcyclohexane, ethylcyclohexane, chlorocyclohexane, dioxane, tetrahydrofuran, diethyl ether, 1,2-dimethoxyethane, decalin, hexane, heptane, toluene, xylene, mesitylene, dichloromethane, 1,2-dichloroethane, 1,1,1-trichloroethane, 1,1,2,2-tetrachloroethane, 1,1,2,2-tetrachloroethylene, chlorobenzene, 1,2-dichlorobenzene and 1,2,4-trichlorobenzene.

In addition, the present specification provides a deuterated aromatic compound prepared by the above-described method.

The deuterated aromatic compound of the present specification includes a substituent selected from the group consisting of a leaving group, a hydroxyl group, a substituted or unsubstituted amine group and a cyano group.

In the deuterated aromatic compound of the present specification, the leaving group can be selected from the group consisting of a halogen group and a boronic acid group.

In the deuterated aromatic compound of the present specification, a deuterated aromatic compound including the substituent selected from the group consisting of a leaving group, a hydroxyl group, a substituted or unsubstituted amine group and a cyano group can be any one of Chemical Formulae 7 to 10:

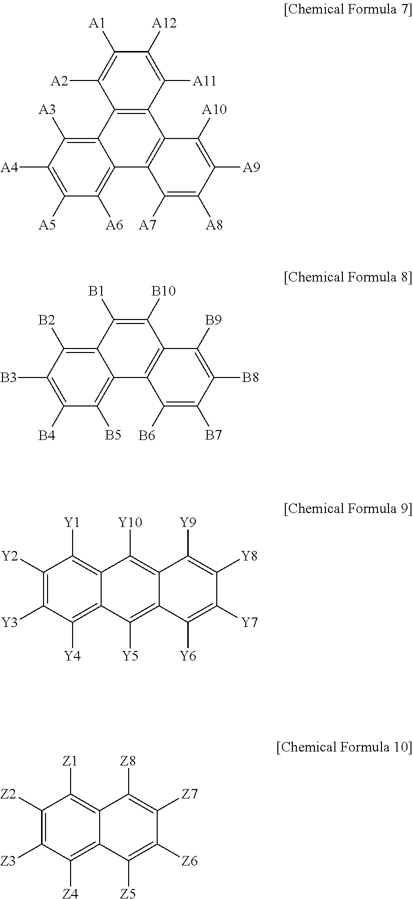

wherein in Chemical Formulae 7 to 10:

at least one of A1 to A12 is deuterium, at least one is a substituent selected from the group consisting of a leaving group, a hydroxyl group, a substituted or unsubstituted amine group and a cyano group, and the others are each independently hydrogen, a leaving group, a hydroxyl group, a substituted or unsubstituted amine group, a cyano group, a substituted or unsubstituted alkyl group, a substituted or unsubstituted cycloalkyl group, a substituted or unsubstituted aryl group, or a substituted or unsubstituted heterocyclic group, or can be bonded to an adjacent group to form a substituted or unsubstituted hydrocarbon ring;

at least one of B1 to B10 is deuterium, at least one is a substituent selected from the group consisting of a leaving group, a hydroxyl group, a substituted or unsubstituted amine group and a cyano group, and the others are each independently hydrogen, a leaving group, a hydroxyl group, a substituted or unsubstituted amine group, a cyano group, a substituted or unsubstituted alkyl group, a substituted or unsubstituted cycloalkyl group, a substituted or unsubstituted aryl group, or a substituted or unsubstituted heterocyclic group, or can be bonded to an adjacent group to form a substituted or unsubstituted hydrocarbon ring;

at least one of Y1 to Y10 is deuterium, at least one is a substituent selected from the group consisting of a leaving group, a hydroxyl group, a substituted or unsubstituted amine group and a cyano group, and the others are each independently hydrogen, a leaving group, a hydroxyl group, a substituted or unsubstituted amine group, a cyano group, a substituted or unsubstituted alkyl group, a substituted or unsubstituted cycloalkyl group, a substituted or unsubstituted aryl group, or a substituted or unsubstituted heterocyclic group, or can be bonded to an adjacent group to form a substituted or unsubstituted hydrocarbon ring; and

at least one of Z1 to Z8 is deuterium, at least one is a substituent selected from the group consisting of a leaving group, a hydroxyl group, a substituted or unsubstituted amine group and a cyano group, and the others are each independently hydrogen, a leaving group, a hydroxyl group; a substituted or unsubstituted amine group, a cyano group, a substituted or unsubstituted alkyl group, a substituted or unsubstituted cycloalkyl group, a substituted or unsubstituted aryl group, or a substituted or unsubstituted heterocyclic group, or can be bonded to an adjacent group to form a substituted or unsubstituted hydrocarbon ring.

Furthermore, the present specification provides an electronic device including the deuterated aromatic compound.

### Advantageous Effects

A production method of a first exemplary embodiment according to the present specification has an advantage in that impurities due to hydrogen gas are not generated.

A production method of a second exemplary embodiment according to the present specification has an advantage in that the deuterium substitution rate is high.

A production method of a third exemplary embodiment according to the present specification has an advantage in that the purity of an obtained compound is high.

A production method of a fourth exemplary embodiment according to the present specification enables a deuterated reaction to be performed under a lower pressure.

A production method of a fifth exemplary embodiment according to the present specification enables a deuterated reaction to be performed at a lower temperature.

### DETAILED DESCRIPTION

Hereinafter, the present specification will be described in detail.

The present specification provides a method for producing a deuterated aromatic compound, the method including: performing a deuterated reaction of an aromatic compound including one or more hydrocarbon aromatic rings using a solution including heavy water, an organic compound which can be hydrolyzed by the heavy water, the aromatic compound, and an organic solvent.

The method for producing a deuterated aromatic compound of the present specification is characterized in that there is no hydrogen supply step.

In the related art, hydrogen gas was supplied in order to activate a metal catalyst, which is a heterogeneous catalyst added to produce a deuterated aromatic compound. When a deuterated reaction is performed by supplying hydrogen, a hydrogenation reaction is performed by hydrogen gas and thus by-products are generated by a side reaction.

In order to remove the generated by-products, a process of increasing the purity through a purification process after the reaction is required, and even though the purification process as described above is performed, the by-products have no difference in melting point and solubility from a target material, so that it is difficult to produce the deuterated aromatic compound with high purity.

The method for producing a deuterated aromatic compound of the present specification has an advantage in that impurities due to hydrogen gas are not generated because a metal catalyst and hydrogen gas for activating the metal catalyst need not be supplied due to the use of an organic compound which can be hydrolyzed by the heavy water instead of the metal catalyst which is a heterogeneous catalyst.

Meanwhile, when a metal catalyst is used during the conventional deuterated reaction, the metal catalyst reacts with a reactive group of a compound to be deuterated, that is, a halogen group, an amine group, a hydroxyl group, a cyano group, and the like, so that in a deuterated reaction using a metal catalyst, the compound to be deuterated has no choice but to be limited to a compound having no reactive group capable of reacting with the metal catalyst, or a reactive group which has low reactivity.

Since an organic compound which can be hydrolyzed by heavy water is used instead of a metal catalyst which is a heterogeneous catalyst in the method for producing a deuterated aromatic compound of the present specification, a compound having a reactive group such as a halogen group, an amine group, a hydroxyl group and a cyano group can also be selected as the compound to be deuterated. Specifically, after a compound, which is an intermediate having a reactive group such as a halogen group, an amine group, a hydroxyl group and a cyano group, is deuterated, a reaction of substituting the reactive group with an additional aromatic substituent can be performed.

The production method according to the present specification has an advantage in that the deuterium substitution rate is high.

The production method according to the present specification has an advantage in that the purity of an obtained compound is high.

The production method according to the present specification enables a deuterated reaction to be performed under a lower pressure.

The production method according to the present specification enables a deuterated reaction to be produced at a lower temperature.

The method for producing a deuterated aromatic compound of the present specification includes: preparing a solution including an aromatic compound including one or more hydrocarbon aromatic rings, heavy water (D₂O), an organic compound which can be hydrolyzed by the heavy water, and an organic solvent.

The preparing of the solution including the aromatic compound including one or more hydrocarbon aromatic rings, heavy water (D₂O), the organic compound which can be hydrolyzed by the heavy water, and the organic solvent can prepare the solution by introducing a solution including an aromatic compound including one or more hydrocarbon aromatic rings, heavy water (D₂O), an organic compound which can be hydrolyzed by the heavy water, and an organic solvent into a reactor, or individually introducing an aromatic compound including one or more hydrocarbon aromatic rings, heavy water (D₂O), an organic compound which can be hydrolyzed by the heavy water, and an organic solvent into a reactor.

In an exemplary embodiment of the present specification, the organic compound which can be hydrolyzed by the heavy water is not particularly limited as long as the organic compound has a reactive group which can be decomposed by heavy water, and the organic compound can include, for example, at least one compound of the following Chemical Formulae 1 to 4:

R1-C(O)OC(O)—R2 [Chemical Formula 1]

R3-S(O₂)OS(O₂)—R4 [Chemical Formula 2]

R5-C(O)O—R6 [Chemical Formula 3]

R7-CONH—R8 [Chemical Formula 4]

wherein in Chemical Formulae 1 to 4, R1 to R8 are the same as or different from each other, and are each independently a monovalent organic group which is unsubstituted or substituted with a halogen group.

In an exemplary embodiment of the present specification, R1 and R2 can be the same substituent.

In an exemplary embodiment of the present specification, R3 and R4 can be the same substituent.

In an exemplary embodiment of the present specification, R5 and R6 can be the same substituent.

In an exemplary embodiment of the present specification, R7 and R8 can be the same substituent.

In an exemplary embodiment of the present specification, R1 to R8 are the same as or different from each other, and can be each independently an alkyl group which is unsubstituted or substituted with a halogen group, or an aryl group which is unsubstituted or substituted with a halogen group.

In an exemplary embodiment of the present specification, R1 to R8 are the same as or different from each other, and can be each independently an alkyl group having 1 to 30 carbon atoms, which is unsubstituted or substituted with a halogen group, or an aryl group having 6 to 50 carbon atoms, which is unsubstituted or substituted with a halogen group.

In an exemplary embodiment of the present specification, R1 to R8 are the same as or different from each other, and can be each independently an alkyl group having 1 to 10 carbon atoms, which is unsubstituted or substituted with a halogen group, or an aryl group having 6 to 20 carbon atoms, which is unsubstituted or substituted with a halogen group.

In an exemplary embodiment of the present specification, R1 to R8 are the same as or different from each other, and can be each independently an alkyl group having 1 to 10 carbon atoms, which is unsubstituted or substituted with a halogen group.

In an exemplary embodiment of the present specification, R1 to R8 are the same as or different from each other, and can be each independently an alkyl group having 1 to 5 carbon atoms, which is unsubstituted or substituted with a halogen group.

In an exemplary embodiment of the present specification, R1 to R8 are the same as or different from each other, and can be each independently a substituent of the following Chemical Formula 5 or 6:

—(CH₂)ₗ(CF₂)ₘ(CF₃)ₙ(CH₃)₁₋ₙ [Chemical Formula 5]

—C(H)ₐ((CH₂)ₗ(CF₂)ₘCF₃)₃₋ₐ [Chemical Formula 6]

wherein in Chemical Formulae 5 and 6, 1 and m are each an integer from 0 to 10, and n and a are each 0 or 1.

In an exemplary embodiment of the present specification, R1 to R8 are the same as or different from each other, and can be each independently the substituent of Chemical Formula 5.

In an exemplary embodiment of the present specification, R1 to R8 are the same as or different from each other, and can be each independently —CF₃, —CH₂CH₃ or —CH₃.

In the method for producing a deuterated aromatic compound of the present specification, the organic compound which can be hydrolyzed by the heavy water includes at least one of trifluoromethanesulfonic anhydride, trifluoroacetic anhydride, acetic anhydride, methanesulfonic anhydride, methyl acetate, ethyl acetate and dimethylacetamide.

In an exemplary embodiment of the present specification, the organic compound which can be hydrolyzed by the heavy water can include trifluoromethanesulfonic anhydride.

In an exemplary embodiment of the present specification, the organic compound which can be hydrolyzed by the heavy water can include trifluoroacetic anhydride.

In an exemplary embodiment of the present specification, the organic compound which can be hydrolyzed by the heavy water can include acetic anhydride.

In an exemplary embodiment of the present specification, the organic compound which can be hydrolyzed by the heavy water can include methanesulfonic anhydride.

In an exemplary embodiment of the present specification, the organic compound which can be hydrolyzed by the heavy water can include trifluoromethanesulfonic anhydride and trifluoroacetic anhydride.

In an exemplary embodiment of the present specification, the organic compound which can be hydrolyzed by the heavy water can include trifluoromethanesulfonic anhydride and acetic anhydride.

In an exemplary embodiment of the present specification, the organic compound which can be hydrolyzed by the heavy water can include methanesulfonic anhydride and trifluoroacetic anhydride.

In an exemplary embodiment of the present specification, the organic compound which can be hydrolyzed by the heavy water can include methanesulfonic anhydride and acetic anhydride.

In an exemplary embodiment of the present specification, the organic compound which can be hydrolyzed by the heavy water can include at least one of the compound of Chemical Formula 1 and the compound of Chemical Formula 2. When at least one of the compound of Chemical Formula 1 and the compound of Chemical Formula 2 is introduced into heavy water, hydrolysis with heavy water easily occurs even at room temperature.

In an exemplary embodiment of the present specification, the organic compound which can be hydrolyzed by the heavy water includes at least one of the compound of Chemical Formula 1 and the compound of Chemical Formula 2, and can further include at least one of the compound of Chemical Formula 3 and the compound of Chemical Formula 4. When the organic compound which can be hydrolyzed by the heavy water includes at least one of the compound of Chemical Formula 1 and the compound of Chemical Formula 2, it is possible to control a temperature occurring due to a hydrolysis reaction which is an exothermic reaction by adding at least one of the compound of Chemical Formula 3 and the compound of Chemical Formula 4 having a relatively slow hydrolysis reaction.

In an exemplary embodiment of the present specification, when the organic compound which can be hydrolyzed by the heavy water includes at least one of the compound of Chemical Formula 3 and the compound of Chemical Formula 4, the organic compound can further include at least one of the compound of Chemical Formula 1 and the compound of Chemical Formula 2. The hydrolysis reaction can be accelerated by adding the compound of Chemical Formulas 1 and the compound of Chemical Formula 2, in which the hydrolysis reaction is relatively easily occurs.

In an exemplary embodiment of the present specification, the organic compound which can be hydrolyzed by the heavy water includes at least one of trifluoromethanesulfonic anhydride, trifluoroacetic anhydride, acetic anhydride and methanesulfonic anhydride, and can further include at least one of methyl acetate, ethyl acetate and dimethylacetamide.

In an exemplary embodiment of the present specification, in the organic compound which can be hydrolyzed by the heavy water, a weight ratio of at least one of the compound of Chemical Formula 3 and the compound of Chemical Formula 4 to at least one of the compound of Chemical Formula 1 and the compound of Chemical Formula 2 can be 100:0 to 0:100, 99:1 to 0:100, 90:10 to 0:100, 80:20 to 0:100, 70:30 to 0:100, 60:40 to 0:100, 50:50 to 0:100, 40:60 to 0:100, 30:70 to 0:100, 20:80 to 0:100, or 10:90 to 0:100.

According to an exemplary embodiment of the present specification, a content of the organic compound which can be hydrolyzed by the heavy water can be 1 mol % or more and 100 mol % or less based on the mole of heavy water. Specifically, according to an exemplary embodiment of the present specification, a content of the organic compound which can be hydrolyzed by the heavy water is not more than the molar equivalent of the heavy water, and is adjusted according to a target material. In this case, there is an advantage in that it is possible to increase the affinity between the aromatic compound and heavy water which are immiscible with each other and a deuterium substitution reactivity is enhanced.

According to an exemplary embodiment of the present specification, a content of the organic solvent can be 1-fold to 40-fold, specifically 3-fold to 15-fold of that of the aromatic compound, based on the weight of an aromatic compound including one or more hydrocarbon aromatic rings to be described below. In this case, the temperature of the entire process can be controlled and the reaction time can be shortened.

In the method for producing a deuterated aromatic compound of the present specification, the performing of the deuterated reaction of the aromatic compound includes:

preparing a solution including an aromatic compound including one or more hydrocarbon aromatic rings, heavy water, an organic compound which can be hydrolyzed by the heavy water, and an organic solvent; and

performing the deuterated reaction of the aromatic compound by heating the solution.

According to an exemplary embodiment of the present specification, the content of the aromatic compound can be 3-fold or more and 100-fold or less based on the mole of the organic compound which can be hydrolyzed. In this case, there is an advantage in that deuterium can be efficiently substituted from an organic compound which can be hydrolyzed by heavy water.

According to an exemplary embodiment of the present specification, the content of heavy water can be 0.1-fold or more and 30-fold or less of the weight of the aromatic compound. In this case, there is an advantage in that deuterium can be efficiently substituted from heavy water.

According to an exemplary embodiment of the present specification, the solution for a deuterated reaction can include an additional deuterium source. The additional deuterium source can be a deuterated aromatic solvent, for example, benzene-d6, toluene-d8, and the like.

According to an exemplary embodiment of the present specification, the content of the additional deuterium source can be 0.1-fold or more and 30-fold or less of the weight of the aromatic compound. In this case, there is an advantage in that the reactivity can be enhanced and the heat generation during the reaction can be reduced.

In the method for producing a deuterated aromatic compound of the present specification, the solution for a deuterated reaction further includes an organic solvent. The organic solvent is not particularly limited as long as the organic solvent can dissolve the aromatic compound, and can be selected according to the used aromatic compound.

In an exemplary embodiment of the present specification, a reaction can also be performed in one phase using an organic solvent which is miscible with heavy water, and a deuterated reaction can be performed at the interface by separating the phase into two phases using an organic solvent which is immiscible with heavy water, as the organic solvent.

Specifically, in the case of a one-phase reaction, an organic compound capable of an excessive amount of hydrolysis is required to maintain a suitable concentration of the entire hydrolyzed organic compound, but in the case of a two-phase reaction, it is possible to reduce the amount of a hydrolyzable organic compound in order to maintain a suitable concentration of the hydrolyzed organic compound. Through this, in the case of the two-phase reaction, the amount of hydrolyzed organic compound can be reduced, so that an increase in purity of the deuterium substitution rate can be induced.

When the number of hydrogens in the aromatic compound is large, an excessive amount of heavy water is required to increase the deuterium substitution rate, but when the deuterated reaction is performed in one phase, the solubility of the aromatic compound deteriorates due to an excessive amount of heavy water, so that reactants are highly likely to be precipitated during or before and after the reaction. In contrast, when the deuterated reaction is performed in two phases, the aromatic compound is dissolved in the organic solvent, and the heavy water and the hydrolyzed organic compound are present while being separated into an aqueous solution layer, so that in the case of two phases, the deuterium substitution rate of the aromatic compound can be increased without a precipitation problem during the reaction even when an excessive amount of heavy water is used.

When the organic solvent is not used, in the case where a certain concentration or more of a hydrolyzed organic compound having deuterium by the hydrolysis reaction of a hydrolyzable organic compound is produced, the hydrolyzed organic compound having deuterium causes heavy water and an aromatic compound which is a target material to be mixed with each other, so that the deuterium substitution reaction is likely to occur.

However, since the organic compound hydrolyzed by heavy water itself is a superacid, an increase in the concentration of the hydrolyzed organic compound tends to cause a side reaction, thereby lowering the purity. In addition, it can also be dangerous in terms of stability to handle a solution containing a large amount of hydrolyzed organic compounds during the work-up process after the reaction.

In contrast, compared to the deuterated reaction without an organic solvent, when an organic solvent is used together, the amount of an organic compound which can be hydrolyzed by heavy water used can be reduced by about 30 to 90%, so that the purity can be increased and the stability can be improved.

When the organic solvent is not used, the concentration of deuterium-substituted trifluoromethanesulfonic acid formed by the hydrolysis reaction of trifluoromethanesulfonic anhydride added as the organic compound which can be hydrolyzed by heavy water is increased, so that the deuterium substitution reaction is likely to occur.

However, since the trifluoromethanesulfonic acid itself is a superacid, an increase in the concentration of the trifluoromethanesulfonic acid tends to cause a side reaction, thereby lowering the purity. Furthermore, it can also be dangerous in terms of stability to handle a solution containing a large amount of trifluoromethanesulfonic acid during the work-up process after the reaction.

In contrast, when the organic solvent is used together, the amount of trifluoromethane sulfonic anhydride used can be reduced by about 30 to 90% compared to the existing amount, so that the purity can be increased and the stability can be improved.

In the method for producing a deuterated aromatic compound of the present specification, the organic solvent is selected from the group consisting of a hydrocarbon chain which is unsubstituted or substituted with a group selected from an alkyl group and a halogen group; an aliphatic hydrocarbon ring which is unsubstituted or substituted with a group selected from an alkyl group and a halogen group; an aromatic hydrocarbon ring which is unsubstituted or substituted with a group selected from an alkyl group and a halogen group; an alkene compound which is unsubstituted or substituted with a group selected from an alkyl group and a halogen group; a straight-chained or branched heterochain; a substituted or unsubstituted aliphatic hetero ring; and a substituted or unsubstituted aromatic hetero ring.

In an exemplary embodiment of the present specification, the organic solvent is selected from the group consisting of an alkyl which is unsubstituted or substituted with a halogen group; a monocyclic or polycyclic cycloalkyl which is unsubstituted or substituted with an alkyl group; a benzene ring which is unsubstituted or substituted with an alkyl group; a substituted or unsubstituted alkyl acetate; alkyl ketone; alkyl sulfoxide; a lactone having 4 to 10 carbon atoms; alkylamide; a glycol having 4 to 10 carbon atoms; dioxane; alkyl ether; an acetic acid which is unsubstituted or substituted with alkoxy.

In the method for producing a deuterated aromatic compound of the present specification, the organic solvent is selected from the group consisting of cyclohexane, methylcyclohexane, ethylcyclohexane, chlorocyclohexane, dioxane, tetrahydrofuran, diethyl ether, 1,2-dimethoxyethane, decalin, hexane, heptane, toluene, xylene, mesitylene, dichloromethane, 1,2-dichloroethane, 1,1,1-trichloroethane, 1,1,2,2-tetrachloroethane, 1,1,2,2-tetrachloroethylene, chlorobenzene, 1,2-dichlorobenzene and 1,2,4-trichlorobenzene.

When the content of the organic solvent is too large, the deuterium substitution rate decreases, and conversely, when the content of the organic solvent is too small, the reactants cannot be dissolved well, and thus the deuterium substitution rate decreases. Preferably, the mass ratio of the organic solvent can be 2-fold to 40-fold, specifically 3-fold to 16-fold, based on the mass of the aromatic compound.

According to an exemplary embodiment of the present specification, the solution is characterized by the fact that the solution does not contain a metal catalyst and an organic compound which can be hydrolyzed by heavy water plays the role thereof instead of the metal catalyst. Through this, problems caused by adding a metal catalyst, for example, the fact that hydrogen gas needs to be supplied, the fact that impurities due to hydrogen gas need to be removed, a fact that the process equipment capable of maintaining and withstanding a high reaction temperature and a high pressure needs to be provided, and the like are solved.

According to an exemplary embodiment of the present specification, the performing of the deuterated reaction of the aromatic compound can be performed by heating the solution.

The performing of the deuterated reaction of the aromatic compound by heating the reactor can be a step of heating the solution at a temperature of 160° C. or less, 150° C. or less, 140° C. or less, 130° C. or less, 120° C. or less, 110° C. or less, 100° C. or less, 90° C. or less, or 80° C. or more, specifically, a temperature of 80° C. or more and 140° C. or less.

When the reactor is heated at a temperature of 80° C. or less, the deuterated reaction rate of the aromatic compound can slow down, so that the deuterium substitution rate of the final compound may not be high. Further, when the reactor is heated sufficiently to 160° C. or more, a large amount of side products which are not desired can be produced.

In this case, the deuterium reaction time is reacted for 1 hour or more after the temperature is completely increased. Specifically, the deuterium reaction time can be reacted for 1 hour or more and 24 hours or less, preferably for 2 hours or more and 18 hours or less, after the temperature in the deuterium reaction is completely increased.

The method for producing a deuterated aromatic compound of the present specification further includes recovering the deuterated aromatic compound after performing the deuteration. The recovering method can be performed by a method known in the art, and is not particularly limited.

The higher the deuterium substitution rate of the recovered deuterated aromatic compound, the better the deuterium substitution rate, and specifically, the deuterium substitution rate of the recovered deuterated aromatic compound can be 50% or more, 60% or more, 70% or more, 80% or more, 85% or more, 90% or more, 91% or more, 92% or more, 93% or more, 94% or more, 95% or more, 96% or more, 97% or more, 98% or more, 99% or more or 100%.

The higher the purity of the recovered deuterated aromatic compound, the better the purity, and specifically, the purity of the recovered deuterated aromatic compound can be 90% or more, 91% or more, 92% or more, 93% or more, 94% or more, 95% or more, 96% or more, 97% or more, 98% or more, 99% or more or 100%.

In an exemplary embodiment of the present specification, the aromatic compound is an aromatic compound including one or more hydrocarbon aromatic rings, and specifically, is an aromatic compound including 1 to 30 hydrocarbon aromatic rings. In this case, the meaning of having one or more hydrocarbon aromatic rings means that there can be one or more hydrocarbon aromatic rings of a monocyclic ring, a polycyclic ring, or a combination thereof, or there can be one or more hydrocarbon aromatic rings (for example, a benzene ring) which are a basic unit. For example, the anthracene ring means one hydrocarbon aromatic ring, or can mean that three benzene rings are linked based on a benzene ring which is a basic unit.

According to an exemplary embodiment of the present specification, the content of the aromatic compound can be 3 wt % or more and 50 wt % or less based on the total weight of the solvent.

In an exemplary embodiment of the present specification, the hydrocarbon aromatic ring can be a substituted or unsubstituted, monocyclic or polycyclic hydrocarbon aromatic ring. For example, the hydrocarbon aromatic ring can be a substituted or unsubstituted benzene ring, a substituted or unsubstituted naphthalene ring, a substituted or unsubstituted anthracene ring, a substituted or unsubstituted triphenylene ring, a substituted or unsubstituted phenanthrene ring, and the like.

In the present specification, the aromatic compound including the hydrocarbon aromatic ring means that an aromatic ring forming a backbone is a hydrocarbon ring, a substituted hydrogen in the corresponding backbone can be substituted with another substituent, and in this case, the type of substituent is not particularly limited. In an exemplary embodiment of the present specification, the aromatic compound can be an anthracene-based compound.

In an exemplary embodiment of the present specification, the aromatic compound can be benzene, toluene, naphthalene, naphthylamine, and the like.

In an exemplary embodiment of the present specification, the aromatic compound can be an anthracene-based compound, and specifically, can be a substituted or unsubstituted anthracene.

In an exemplary embodiment of the present specification, the aromatic compound participating in the deuterated reaction can include a compound of the following Chemical Formula A. By the deuterated reaction, at least one hydrogen of the selected compounds is substituted with deuterium.

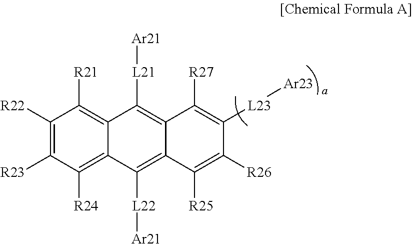

In Chemical Formula A:

L21 to L23 are the same as or different from each other, and are each independently a direct bond, or a substituted or unsubstituted arylene group, or a substituted or unsubstituted heteroarylene group;

R21 to R27 are the same as or different from each other, and are each independently hydrogen, a substituted or unsubstituted alkyl group, a substituted or unsubstituted cycloalkyl group, a substituted or unsubstituted silyl group, a substituted or unsubstituted aryl group, or a substituted or unsubstituted heteroaryl group;

Ar21 to Ar23 are the same as or different from each other, and are each independently a substituted or unsubstituted aryl group, or a substituted or unsubstituted heteroaryl group; and

a is 0 or 1.

In an exemplary embodiment of the present specification, the aromatic compound participating in the deuterated reaction can be any one of the following Chemical Formulae 7 to 10. By the deuterated reaction, at least one hydrogen of the selected compounds is substituted with deuterium.

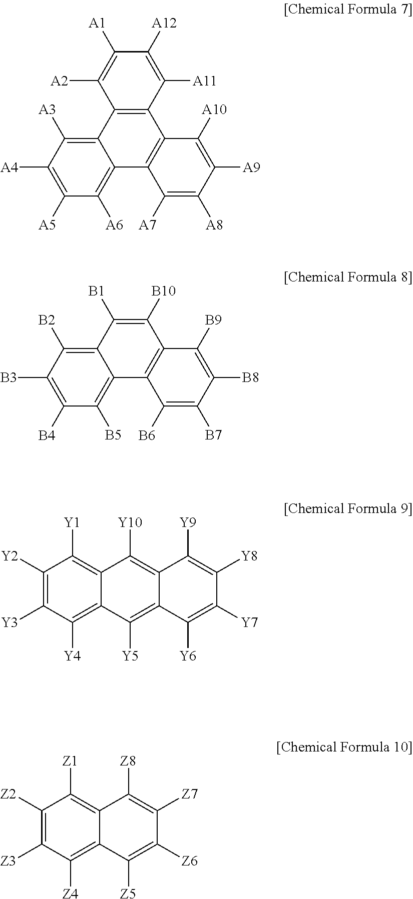

In Chemical Formulae 7 to 10:

A1 to A12 are each independently hydrogen, a leaving group, a hydroxyl group, a substituted or unsubstituted amine group, a cyano group, a substituted or unsubstituted alkyl group, a substituted or unsubstituted cycloalkyl group, a substituted or unsubstituted aryl group, or a substituted or unsubstituted heterocyclic group, or can be bonded to an adjacent group to form a substituted or unsubstituted hydrocarbon ring;

B1 to B10 are each independently hydrogen, a leaving group, a hydroxyl group, a substituted or unsubstituted amine group, a cyano group, a substituted or unsubstituted alkyl group, a substituted or unsubstituted cycloalkyl group, a substituted or unsubstituted aryl group, or a substituted or unsubstituted heterocyclic group, or can be bonded to an adjacent group to form a substituted or unsubstituted hydrocarbon ring;

Y1 to Y10 are each independently hydrogen, a leaving group, a hydroxyl group, a substituted or unsubstituted amine group, a cyano group, a substituted or unsubstituted alkyl group, a substituted or unsubstituted cycloalkyl group, a substituted or unsubstituted aryl group, or a substituted or unsubstituted heterocyclic group, or can be bonded to an adjacent group to form a substituted or unsubstituted hydrocarbon ring; and

Z1 to Z8 are each independently hydrogen, a leaving group, a hydroxyl group, a substituted or unsubstituted amine group, a cyano group, a substituted or unsubstituted alkyl group, a substituted or unsubstituted cycloalkyl group, a substituted or unsubstituted aryl group, or a substituted or unsubstituted heterocyclic group, or can be bonded to an adjacent group to form a substituted or unsubstituted hydrocarbon ring.

Examples of the substituents in the present specification will be described below, but are not limited thereto.

The term “substitution” means that a hydrogen atom bonded to a carbon atom of a compound is changed into another substituent, and a position to be substituted is not limited as long as the position is a position at which the hydrogen atom is substituted, that is, a position at which the substituent can be substituted, and when two or more are substituted, the two or more substituents can be the same as or different from each other.

In the present specification, the term “substituted or unsubstituted” means being substituted with one or two or more substituents selected from the group consisting of a halogen group, a cyano group, a nitro group, a hydroxy group, an amine group, a silyl group, a boron group, an alkoxy group, an alkyl group, a cycloalkyl group, an aryl group, and a heterocyclic group, being substituted with a substituent to which two or more substituents among the above-exemplified substituents are linked, or having no substituent. For example, “the substituent to which two or more substituents are linked” can be a biphenyl group. That is, the biphenyl group can also be an aryl group, and can be interpreted as a substituent to which two phenyl groups are linked.

In the present specification, the “adjacent” group can mean a substituent substituted with an atom directly linked to an atom in which the corresponding substituent is substituted, a substituent disposed to be sterically closest to the corresponding substituent, or another substituent substituted with an atom in which the corresponding substituent is substituted. For example, two substituents substituted at the ortho position in a benzene ring and two substituents substituted with the same carbon in an aliphatic ring can be interpreted as groups which are “adjacent” to each other. In addition, substituents (four in total) linked to two consecutive carbons in an aliphatic ring can be interpreted as “adjacent” groups.

In the present specification, the “adjacent groups are bonded to each other to form a hydrocarbon ring” among the substituents means that a substituent is bonded to an adjacent group to form a substituted or unsubstituted hydrocarbon ring.

In the present specification, “a five-membered or six-membered ring formed by bonding adjacent groups” means that a ring including a substituent participating in the ring formation is five-membered or six-membered. It is possible to include an additional ring fused to the ring including the substituent participating in the ring formation.

In the present specification, examples of a halogen group include fluorine (—F), chlorine (—Cl), bromine (—Br) or iodine (—I).

In the present specification, a silyl group can be a chemical formula of —SiYₐY_{b}Y_{c}, and the Yₐ, Y_{b}, and Y_{c} can be each hydrogen, a substituted or unsubstituted alkyl group, or a substituted or unsubstituted aryl group. Specific examples of the silyl group include a trimethylsilyl group, a triethylsilyl group, a tert-butyldimethylsilyl group, a vinyldimethylsilyl group, a propyldimethylsilyl group, a triphenylsilyl group, a diphenylsilyl group, a phenylsilyl group, and the like, but are not limited thereto.

In the present specification, a boron group can be a chemical formula of —BY_{d}Yₑ, and the Y_{d} and Yₑ can be each hydrogen, a substituted or unsubstituted alkyl group, or a substituted or unsubstituted aryl group. Specific examples of the boron group include a trimethylboron group, a triethylboron group, a tert-butyldimethylboron group, a triphenylboron group, a phenylboron group, and the like, but are not limited thereto.

In the present specification, the alkyl group can be straight-chained or branched, and the number of carbon atoms thereof is not particularly limited, but is preferably 1 to 60. According to an exemplary embodiment, the number of carbon atoms of the alkyl group is 1 to 30. According to another exemplary embodiment, the number of carbon atoms of the alkyl group is 1 to 20. According to still another exemplary embodiment, the number of carbon atoms of the alkyl group is 1 to 10. Specific examples of the alkyl group include a methyl group, an ethyl group, a propyl group, an n-propyl group, an isopropyl group, a butyl group, an n-butyl group, an isobutyl group, a tert-butyl group, a pentyl group, an n-pentyl group, a hexyl group, an n-hexyl group, a heptyl group, an n-heptyl group, an octyl group, an n-octyl group, and the like, but are not limited thereto.

In the present specification, the alkoxy group can be straight-chained, branched, or cyclic. The number of carbon atoms of the alkoxy group is not particularly limited, but is preferably 1 to 20. Specific examples thereof include methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, and the like, but are not limited thereto.

Substituents including an alkyl group, an alkoxy group, and other alkyl group moieties described in the present specification include both a straight-chained form and a branched form.

In the present specification, a cycloalkyl group is not particularly limited, but has preferably 3 to 60 carbon atoms, and according to an exemplary embodiment, the number of carbon atoms of the cycloalkyl group is 3 to 30. According to another exemplary embodiment, the number of carbon atoms of the cycloalkyl group is 3 to 20. According to yet another exemplary embodiment, the number of carbon atoms of the cycloalkyl group is 3 to 6. Specific examples thereof include a cyclopropyl group, a cyclobutyl group, a cyclopentyl group, a cyclohexyl group, a cycloheptyl group, a cyclooctyl group, and the like, but are not limited thereto.

In the present specification, an aryl group is not particularly limited, but has preferably 6 to 60 carbon atoms, and can be a monocyclic aryl group or a polycyclic aryl group. According to an exemplary embodiment, the number of carbon atoms of the aryl group is 6 to 39. According to an exemplary embodiment, the number of carbon atoms of the aryl group is 6 to 30. Examples of the monocyclic aryl group include a phenyl group, a biphenyl group, a terphenyl group, a quaterphenyl group, and the like, but are not limited thereto. Examples of the polycyclic aryl group include a naphthyl group, an anthracenyl group, a phenanthrenyl group, a pyrenyl group, a perylenyl group, a triphenyl group, a chrysenyl group, a fluorenyl group, a triphenylenyl group, and the like, but are not limited thereto.

In the present specification, a fluorene group can be substituted, and two substituents can be bonded to each other to form a spiro structure.

When the fluorene group is substituted, the fluorene group can be a spirofluorene group such as

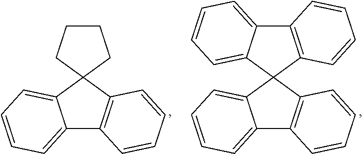

and a substituted fluorene group such as

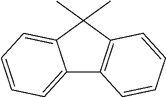

(a 9,9-dimethylfluorene group) and

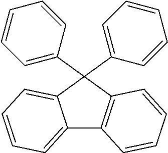 [SMILES: c1ccc(C2(c3ccccc3)c3ccccc3-c3ccccc32)cc1]

(a 9,9-diphenylfluorene group). However, the substituent is not limited thereto.

In the present specification, a heterocyclic group is a cyclic group including one or more of N, O, P, S, Si, and Se as a heteroatom, and the number of carbon atoms thereof is not particularly limited, but is preferably 2 to 60. According to an exemplary embodiment, the number of carbon atoms of the heterocyclic group is 2 to 36. Examples of the heterocyclic group include a pyridine group, a pyrrole group, a pyrimidine group, a quinoline group, a pyridazine group, a furan group, a thiophene group, an imidazole group, a pyrazole group, a dibenzofuran group, a dibenzothiophene group, a carbazole group, a benzocarbazole group, a benzonaphthofuran group, a benzonaphthothiophene group, an indenocarbazole group, an indolocarbazole group, and the like, but are not limited thereto.

In the present specification, the above-described description on the heterocyclic group can be applied to a heteroaryl group except that the heteroaryl group is aromatic.

In the present specification, an amine group can be selected from the group consisting of —NH₂, an alkylamine group, an N-alkylarylamine group, an arylamine group, an N-arylheteroarylamine group, an N-alkylheteroarylamine group, and a heteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, an anthracenylamine group, a 9-methyl-anthracenylamine group, a diphenylamine group, an N-phenylnaphthylamine group, a ditolylamine group, an N-phenyltolylamine group, a triphenylamine group, an N-phenylbiphenylamine group, an N-phenylnaphthylamine group, an N-biphenylnaphthylamine group, an N-naphthylfluorenylamine group, an N-phenylphenanthrenylamine group, an N-biphenylphenanthrenylamine group, an N-phenylfluorenylamine group, an N-phenyl terphenylamine group, an N-phenanthrenylfluorenylamine group, an N-biphenylfluorenylamine group, and the like, but are not limited thereto.

In the present specification, an N-alkylarylamine group means an amine group in which an alkyl group and an aryl group are substituted with N of the amine group.

In the present specification, an N-arylheteroarylamine group means an amine group in which an aryl group and a heteroaryl group are substituted with N of the amine group.

In the present specification, an N-alkylheteroarylamine group means an amine group in which an alkyl group and a heteroaryl group are substituted with N of the amine group.

In the present specification, an alkyl group, an aryl group, and a heteroaryl group in an alkylamine group, an N-alkylarylamine group, an arylamine group, an N-arylheteroarylamine group, an N-alkylheteroarylamine group, and a heteroarylamine group are each the same as the above-described examples of the alkyl group, the aryl group, and the heteroaryl group.

In an exemplary embodiment of the present specification, the aromatic compound can be any one of the following structures:

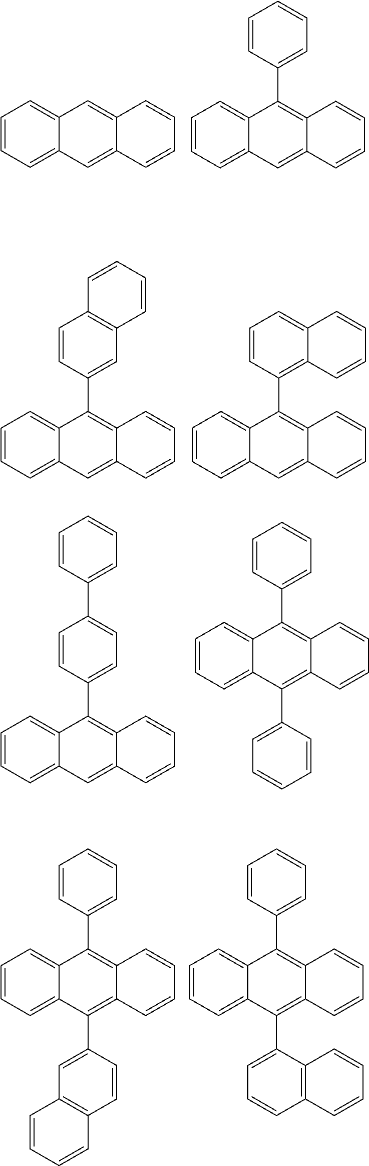

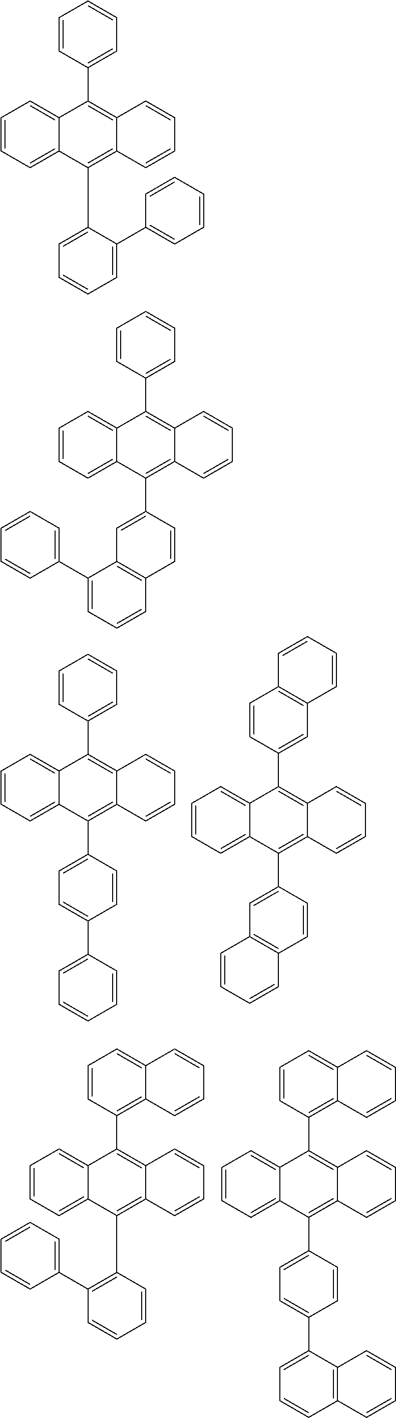

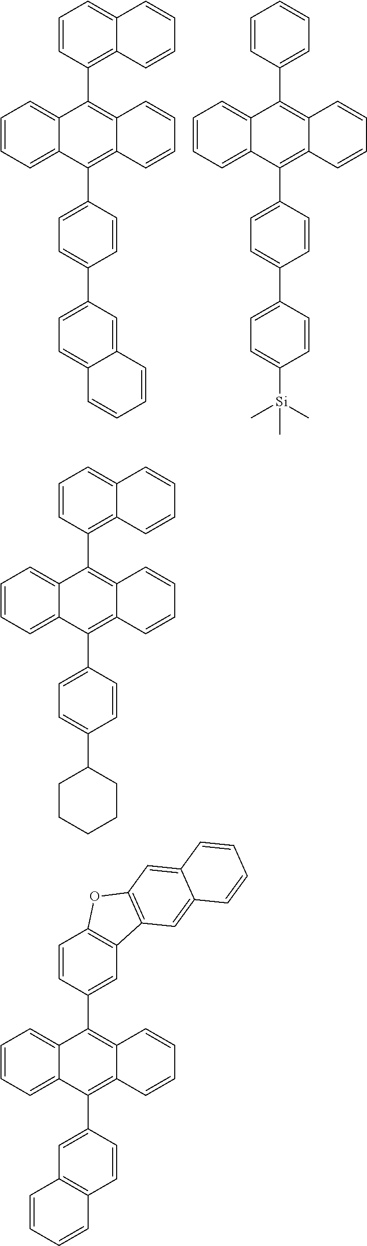

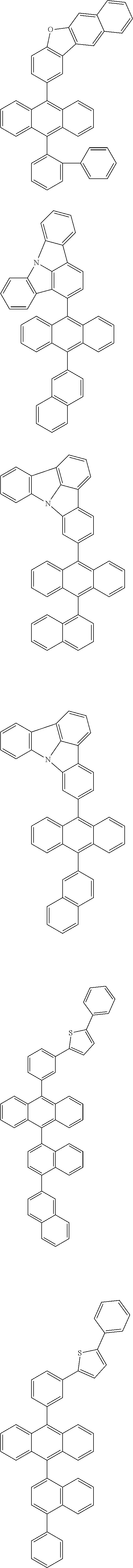

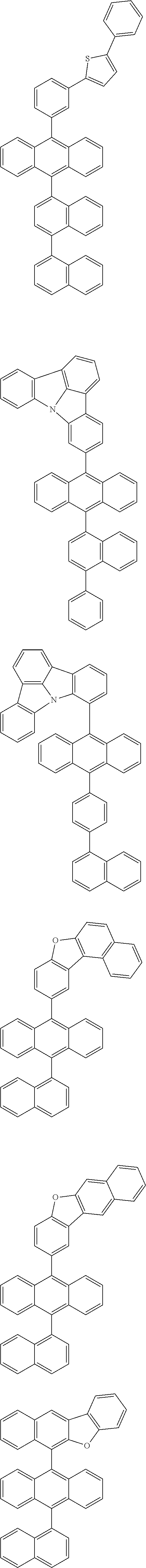 [SMILES: c1ccc(-c2ccc(-c3cccc(-c4c5ccccc5c(-c5ccc(-c6cccc7ccccc67)c6ccccc56)c5ccccc45)c3)s2)cc1 ; c1ccc(-c2ccc(-c3c4ccccc4c(-c4ccc5c6cccc7c8ccccc8n(c5c4)c76)c4ccccc34)c3ccccc23)cc1 ; c1ccc2c(-c3ccc(-c4c5ccccc5c(-c5cccc6c7cccc8c9ccccc9n(c56)c87)c5ccccc45)cc3)cccc2c1 ; c1ccc2c(-c3c4ccccc4c(-c4ccc5oc6ccc7ccccc7c6c5c4)c4ccccc34)cccc2c1 ; c1ccc2cc3c(cc2c1)oc1ccc(-c2c4ccccc4c(-c4cccc5ccccc45)c4ccccc24)cc13 ; c1ccc2c(-c3c4ccccc4c(-c4c5ccccc5cc5c4oc4ccccc45)c4ccccc34)cccc2c1]

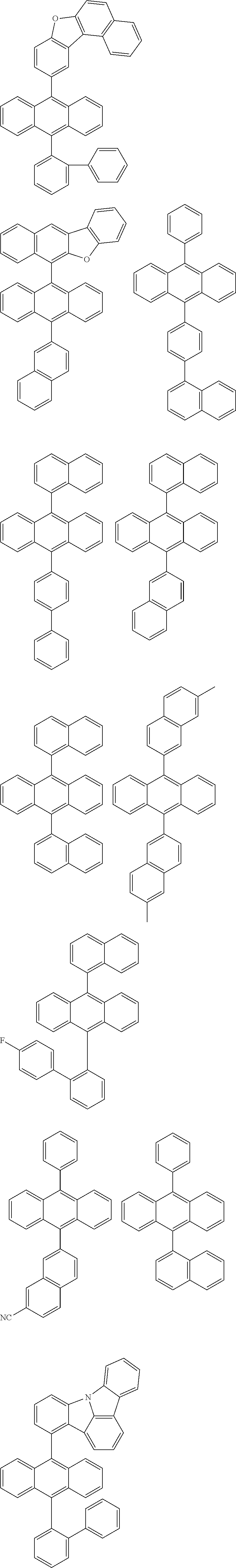

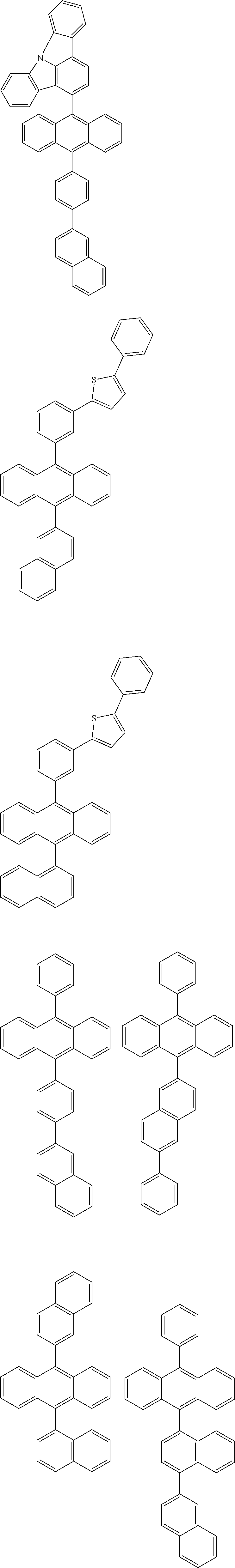

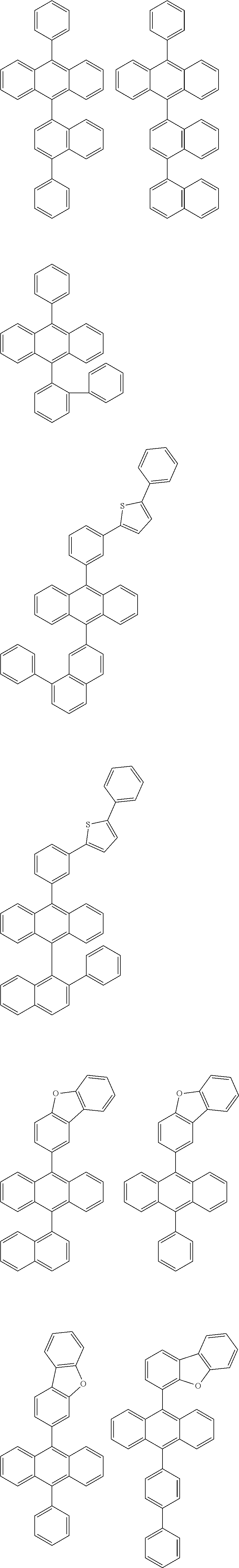

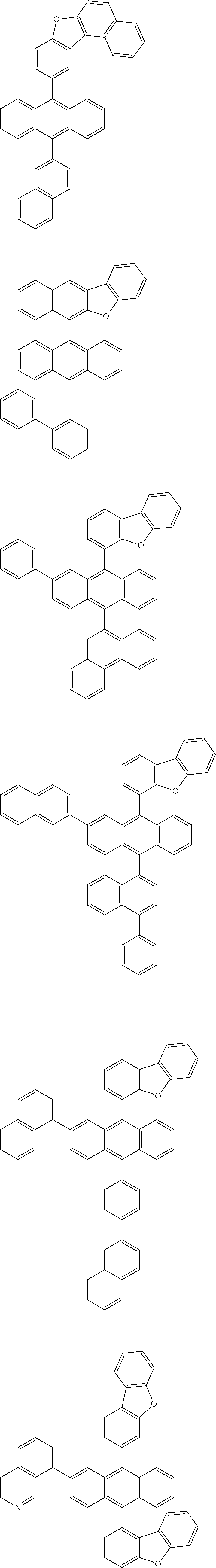 [SMILES: c1ccc2cc(-c3c4ccccc4c(-c4ccc5oc6ccc7ccccc7c6c5c4)c4ccccc34)ccc2c1 ; c1ccc(-c2ccccc2-c2c3ccccc3c(-c3c4ccccc4cc4c3oc3ccccc34)c3ccccc23)cc1 ; c1ccc(-c2ccc3c(-c4cc5ccccc5c5ccccc45)c4ccccc4c(-c4cccc5c4oc4ccccc45)c3c2)cc1 ; c1ccc(-c2ccc(-c3c4ccccc4c(-c4cccc5c4oc4ccccc45)c4cc(-c5ccc6ccccc6c5)ccc34)c3ccccc23)cc1 ; c1ccc2cc(-c3ccc(-c4c5ccccc5c(-c5cccc6c5oc5ccccc56)c5cc(-c6cccc7ccccc67)ccc45)cc3)ccc2c1 ; c1cc(-c2ccc3c(-c4cccc5oc6ccccc6c45)c4ccccc4c(-c4ccc5c(c4)oc4ccccc45)c3c2)c2cnccc2c1]

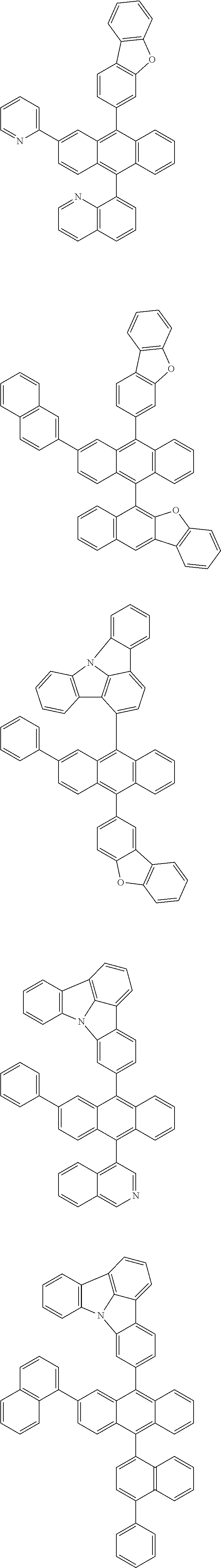 [SMILES: c1ccc(-c2ccc3c(-c4cccc5cccnc45)c4ccccc4c(-c4ccc5c(c4)oc4ccccc45)c3c2)nc1 ; c1ccc2cc(-c3ccc4c(-c5c6ccccc6cc6c5oc5ccccc56)c5ccccc5c(-c5ccc6c(c5)oc5ccccc56)c4c3)ccc2c1 ; c1ccc(-c2ccc3c(-c4ccc5oc6ccccc6c5c4)c4ccccc4c(-c4ccc5c6ccccc6n6c7ccccc7c4c56)c3c2)cc1 ; c1ccc(-c2ccc3c(-c4cncc5ccccc45)c4ccccc4c(-c4ccc5c6cccc7c8ccccc8n(c5c4)c76)c3c2)cc1 ; c1ccc(-c2ccc(-c3c4ccccc4c(-c4ccc5c6cccc7c8ccccc8n(c5c4)c76)c4cc(-c5cccc6ccccc56)ccc34)c3ccccc23)cc1]

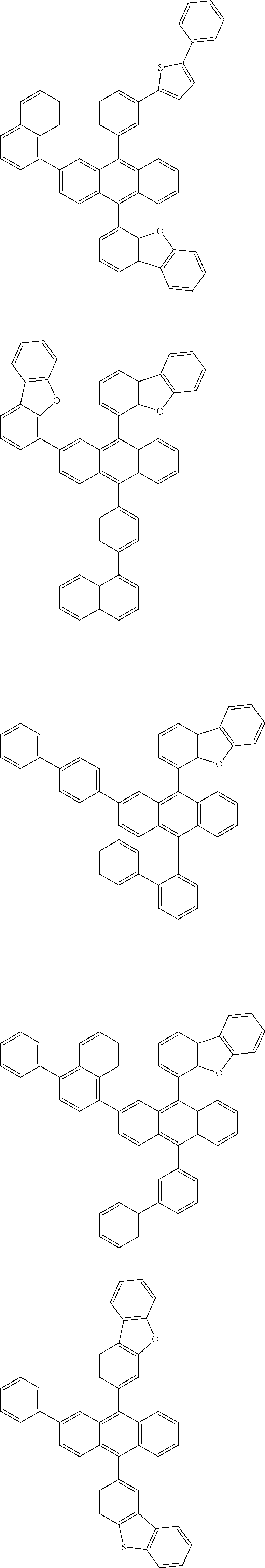 [SMILES: c1ccc(-c2ccc(-c3cccc(-c4c5ccccc5c(-c5cccc6c5oc5ccccc56)c5ccc(-c6cccc7ccccc67)cc45)c3)s2)cc1 ; c1ccc2c(-c3ccc(-c4c5ccccc5c(-c5cccc6c5oc5ccccc56)c5cc(-c6cccc7c6oc6ccccc67)ccc45)cc3)cccc2c1 ; c1ccc(-c2ccc(-c3ccc4c(-c5ccccc5-c5ccccc5)c5ccccc5c(-c5cccc6c5oc5ccccc56)c4c3)cc2)cc1 ; c1ccc(-c2cccc(-c3c4ccccc4c(-c4cccc5c4oc4ccccc45)c4cc(-c5ccc(-c6ccccc6)c6ccccc56)ccc34)c2)cc1 ; c1ccc(-c2ccc3c(-c4ccc5sc6ccccc6c5c4)c4ccccc4c(-c4ccc5c(c4)oc4ccccc45)c3c2)cc1]

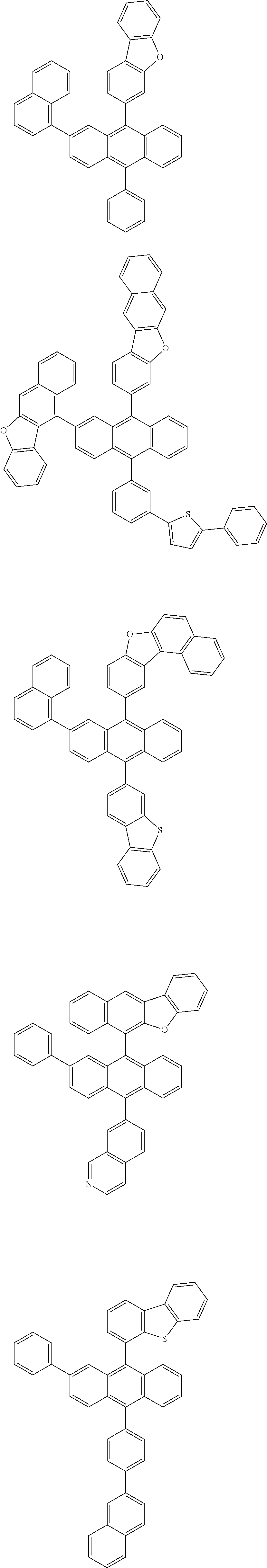

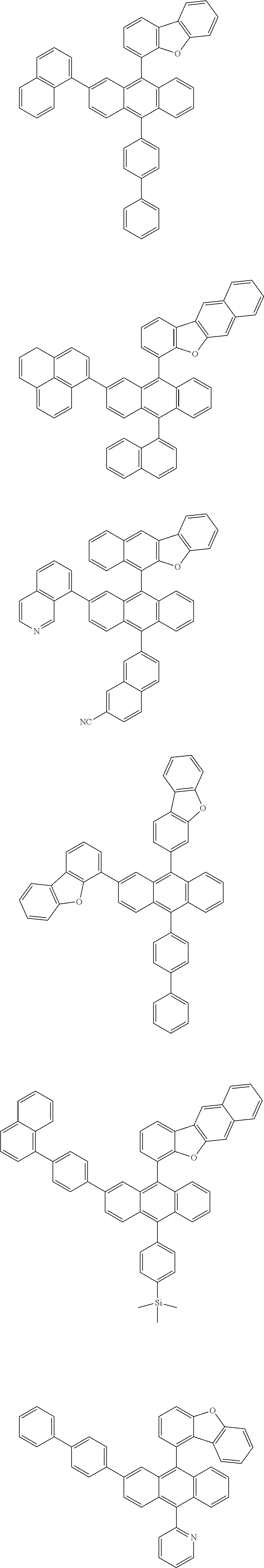

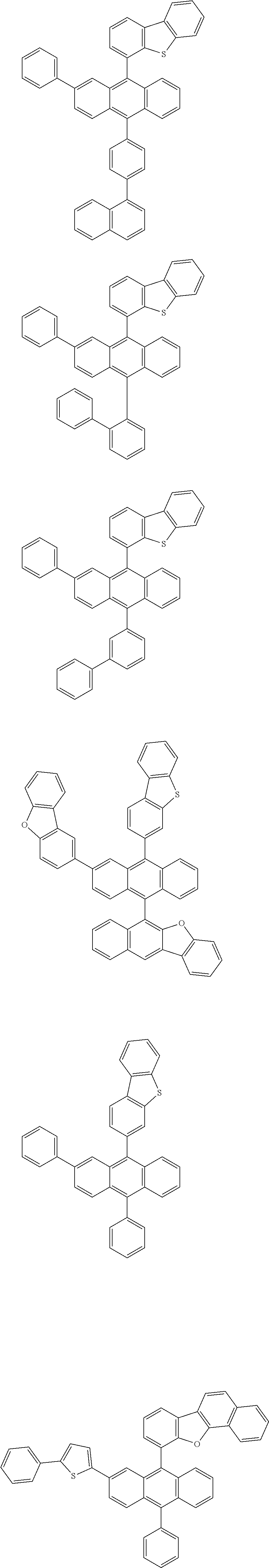 [SMILES: c1ccc(-c2ccc3c(-c4ccc(-c5cccc6ccccc56)cc4)c4ccccc4c(-c4cccc5c4sc4ccccc45)c3c2)cc1 ; c1ccc(-c2ccc3c(-c4ccccc4-c4ccccc4)c4ccccc4c(-c4cccc5c4sc4ccccc45)c3c2)cc1 ; c1ccc(-c2cccc(-c3c4ccccc4c(-c4cccc5c4sc4ccccc45)c4cc(-c5ccccc5)ccc34)c2)cc1 ; c1ccc2c(-c3c4ccccc4c(-c4ccc5c(c4)sc4ccccc45)c4cc(-c5ccc6oc7ccccc7c6c5)ccc34)c3oc4ccccc4c3cc2c1 ; c1ccc(-c2ccc3c(-c4ccccc4)c4ccccc4c(-c4ccc5c(c4)sc4ccccc45)c3c2)cc1 ; c1ccc(-c2ccc(-c3ccc4c(-c5ccccc5)c5ccccc5c(-c5cccc6c5oc5c7ccccc7ccc65)c4c3)s2)cc1]

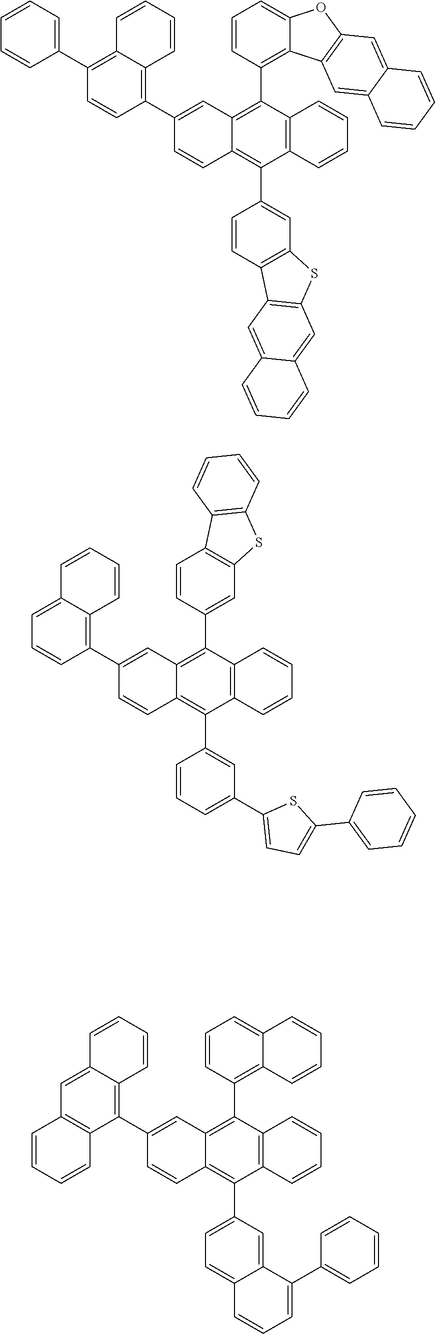

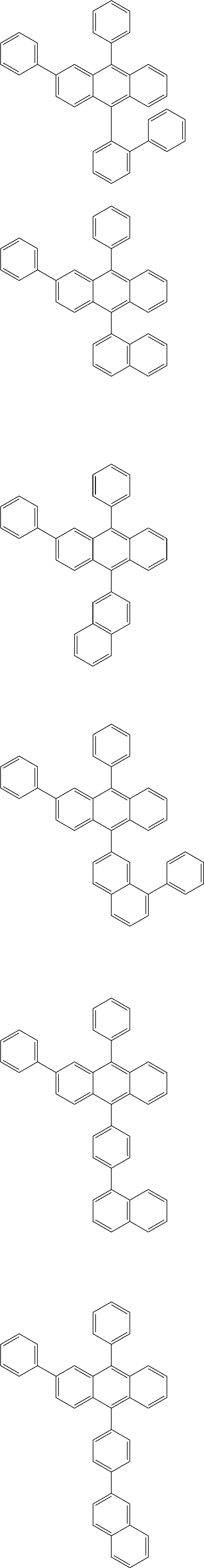

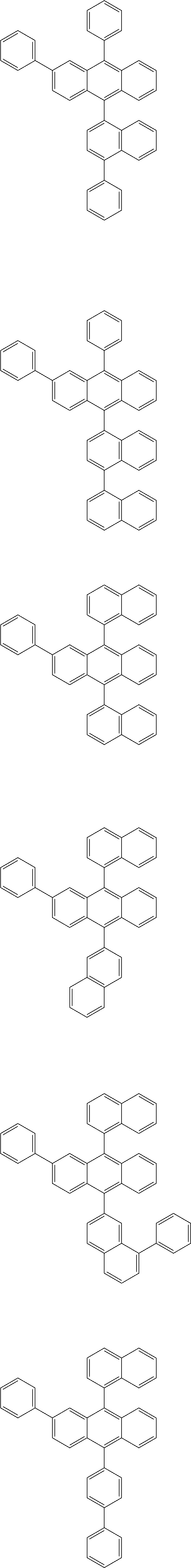

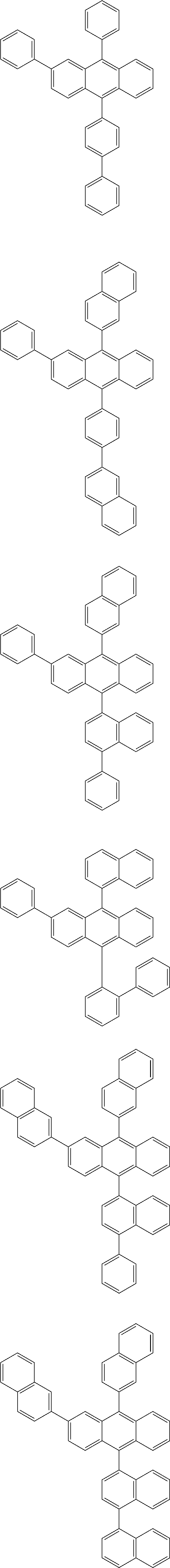

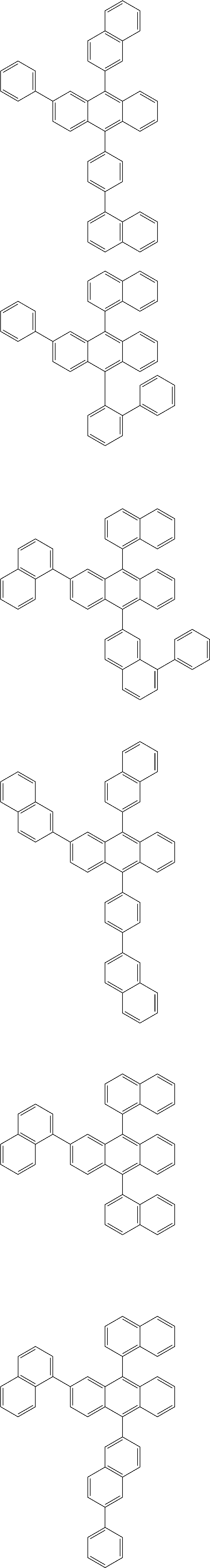

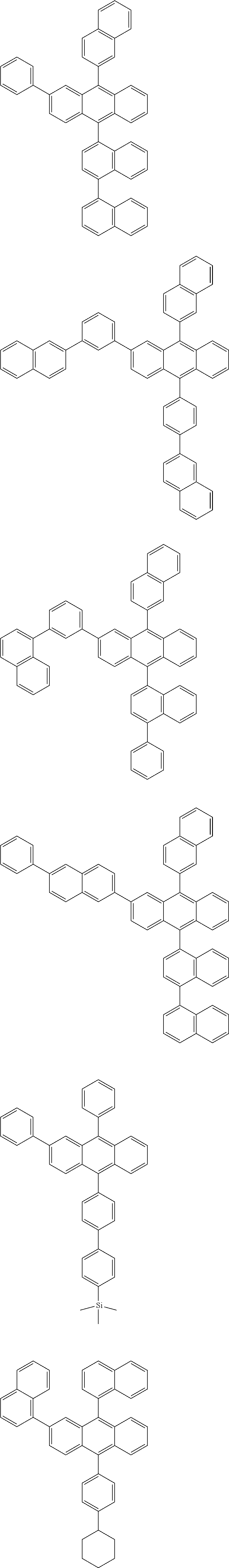

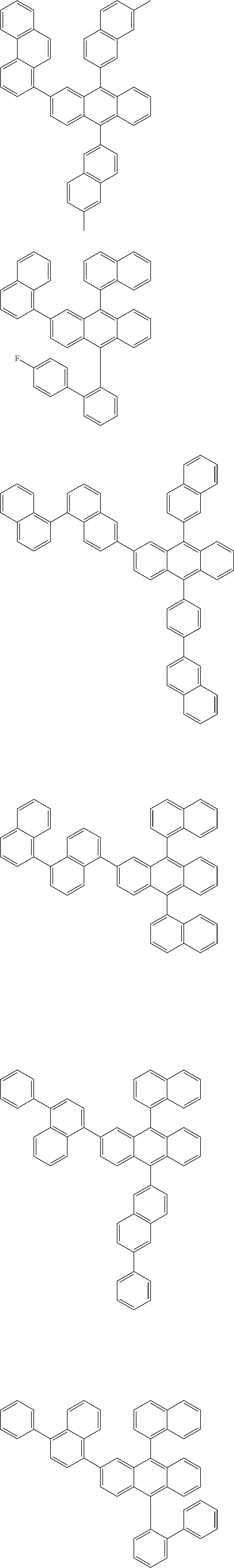

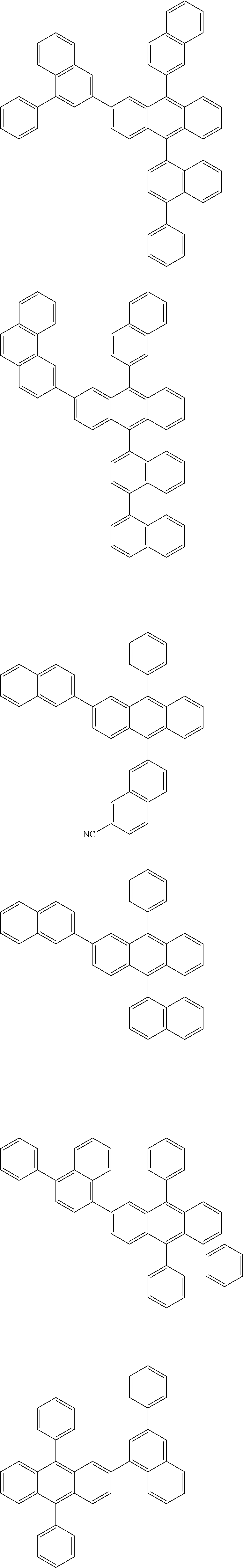

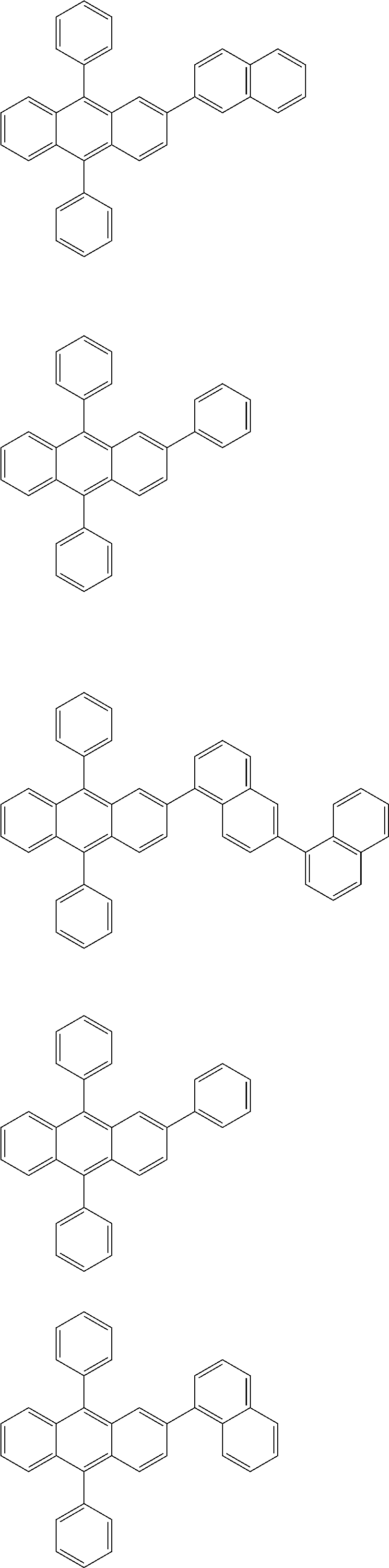

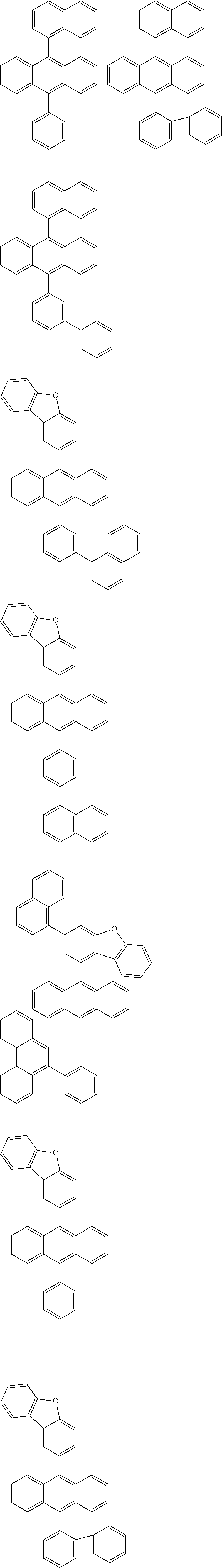

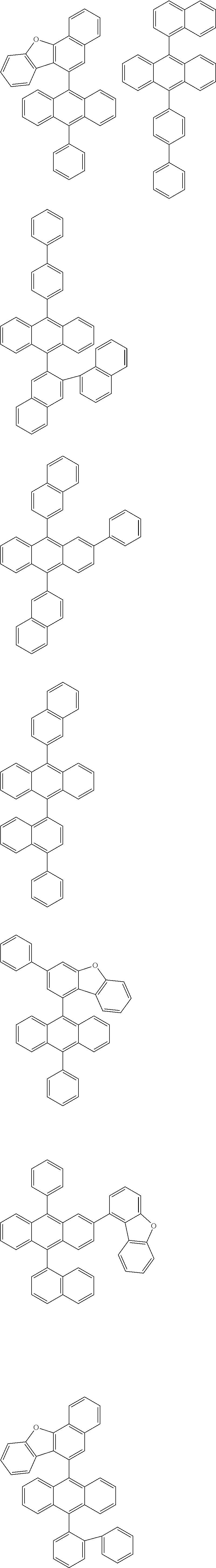

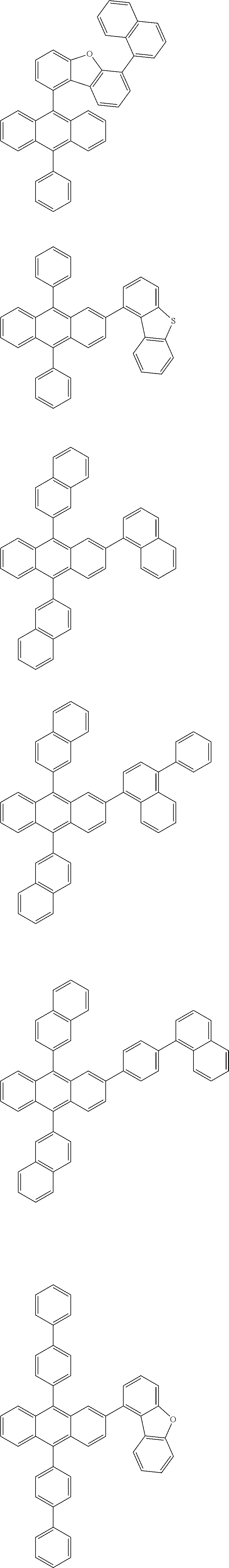

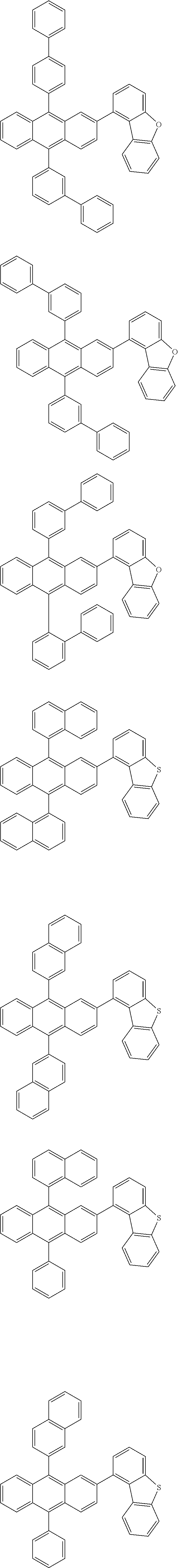

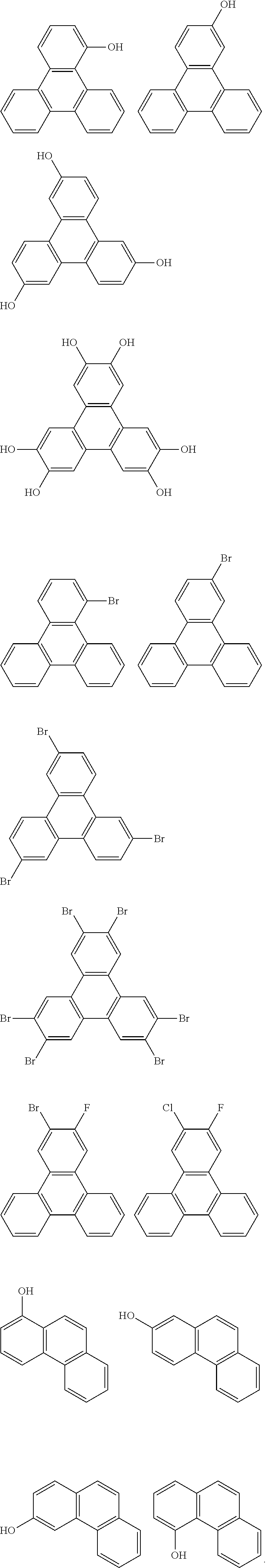

The method for producing a deuterated aromatic compound of the present specification can further include substituting the internal air of the reactor with nitrogen or an inert gas.

The present specification provides a deuterated reaction composition including an aromatic compound including one or more hydrocarbon aromatic rings, heavy water, an organic compound which can be hydrolyzed by the heavy water, and an organic solvent.

For the deuterated reaction composition, the description on the solution in the above-described production method can be cited.

The present specification provides a deuterated aromatic compound produced by the above-described method.

In an exemplary embodiment of the present specification, the deuterated aromatic compound means an aromatic compound which is substituted with at least one or more deuterium.

In an exemplary embodiment of the present specification, the deuterated aromatic compound includes a substituent selected from the group consisting of a leaving group, a hydroxyl group, a substituted or unsubstituted amine group and a cyano group. In the present specification, the compound including the leaving group can be an intermediate of a final compound of organic synthesis, and the leaving group means a reaction group which is left based on the final compound, or is chemically modified by being bonded to other reactants. Thus, for the leaving group, the type of leaving group and the position to which the leaving group is bonded are determined by the method of organic synthesis and the position of the substituent of the final compound.

In the deuterated aromatic compound of the present specification, the leaving group can be selected from the group consisting of a halogen group and a boronic acid group.

In the deuterated aromatic compound of the present specification, a deuterated aromatic compound including the substituent selected from the group consisting of a leaving group, a hydroxyl group, a substituted or unsubstituted amine group and a cyano group can be any one compound of Chemical Formulae 7 to 10:

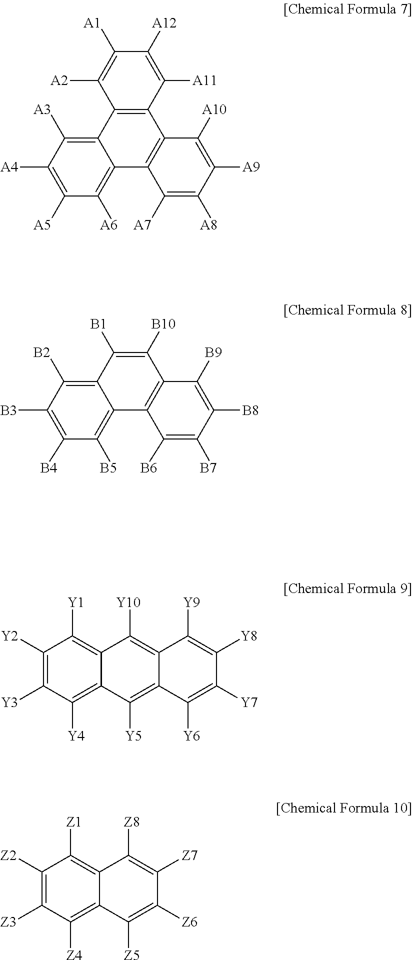

wherein in Chemical Formulae 7 to 10:

at least one of A1 to A12 is deuterium, at least one is a substituent selected from the group consisting of a leaving group, a hydroxyl group, a substituted or unsubstituted amine group and a cyano group, and the others are each independently hydrogen, a leaving group, a hydroxyl group, a substituted or unsubstituted amine group, a cyano group, a substituted or unsubstituted alkyl group, a substituted or unsubstituted cycloalkyl group, a substituted or unsubstituted aryl group, or a substituted or unsubstituted heterocyclic group, or can be bonded to an adjacent group to form a substituted or unsubstituted hydrocarbon ring;

at least one of B1 to B10 is deuterium, at least one is a substituent selected from the group consisting of a leaving group, a hydroxyl group, a substituted or unsubstituted amine group and a cyano group, and the others are each independently hydrogen, a leaving group, a hydroxyl group, a substituted or unsubstituted amine group, a cyano group, a substituted or unsubstituted alkyl group, a substituted or unsubstituted cycloalkyl group, a substituted or unsubstituted aryl group, or a substituted or unsubstituted heterocyclic group, or can be bonded to an adjacent group to form a substituted or unsubstituted hydrocarbon ring;

at least one of Y1 to Y10 is deuterium, at least one is a substituent selected from the group consisting of a leaving group, a hydroxyl group, a substituted or unsubstituted amine group and a cyano group, and the others are each independently hydrogen, a leaving group, a hydroxyl group, a substituted or unsubstituted amine group, a cyano group, a substituted or unsubstituted alkyl group, a substituted or unsubstituted cycloalkyl group, a substituted or unsubstituted aryl group, or a substituted or unsubstituted heterocyclic group, or can be bonded to an adjacent group to form a substituted or unsubstituted hydrocarbon ring; and

at least one of Z1 to Z8 is deuterium, at least one is a substituent selected from the group consisting of a leaving group, a hydroxyl group, a substituted or unsubstituted amine group and a cyano group, and the others are each independently hydrogen, a leaving group, a hydroxyl group, a substituted or unsubstituted amine group, a cyano group, a substituted or unsubstituted alkyl group, a substituted or unsubstituted cycloalkyl group, a substituted or unsubstituted aryl group, or a substituted or unsubstituted heterocyclic group, or can be bonded to an adjacent group to form a substituted or unsubstituted hydrocarbon ring.

The compounds of Chemical Formulae 7 to 10 each have a substituent selected from the group consisting of a leaving group, a hydroxyl group, a substituted or unsubstituted amine group and a cyano group.

A deuterated aromatic compound including the substituent selected from the group consisting of a leaving group, a hydroxyl group, a substituted or unsubstituted amine group and a cyano group is any one compound of the following compounds, and the compounds having the structure are each substituted with one or more deuteriums:

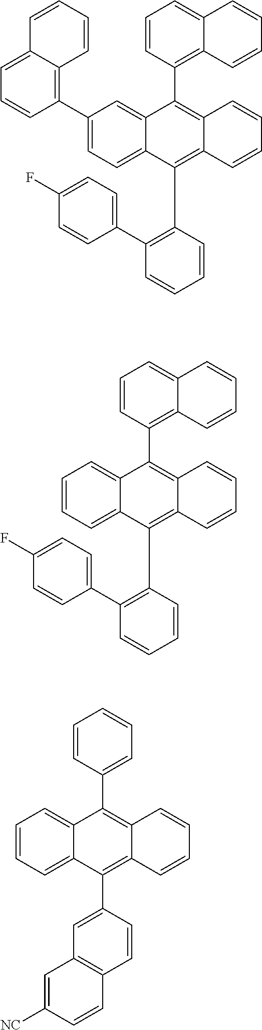 [SMILES: Fc1ccc(-c2ccccc2-c2c3ccccc3c(-c3cccc4ccccc34)c3cc(-c4cccc5ccccc45)ccc23)cc1 ; Fc1ccc(-c2ccccc2-c2c3ccccc3c(-c3cccc4ccccc34)c3ccccc23)cc1 ; N#Cc1ccc2ccc(-c3c4ccccc4c(-c4ccccc4)c4ccccc34)cc2c1]

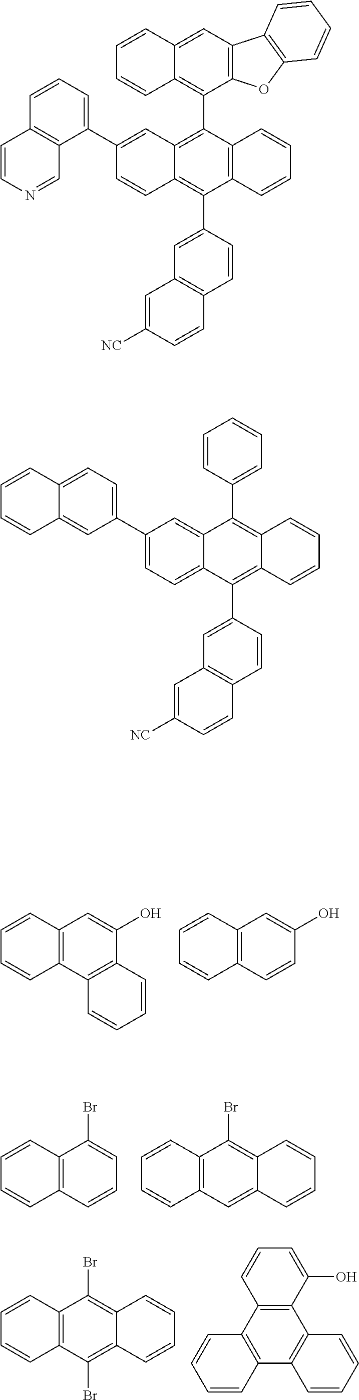

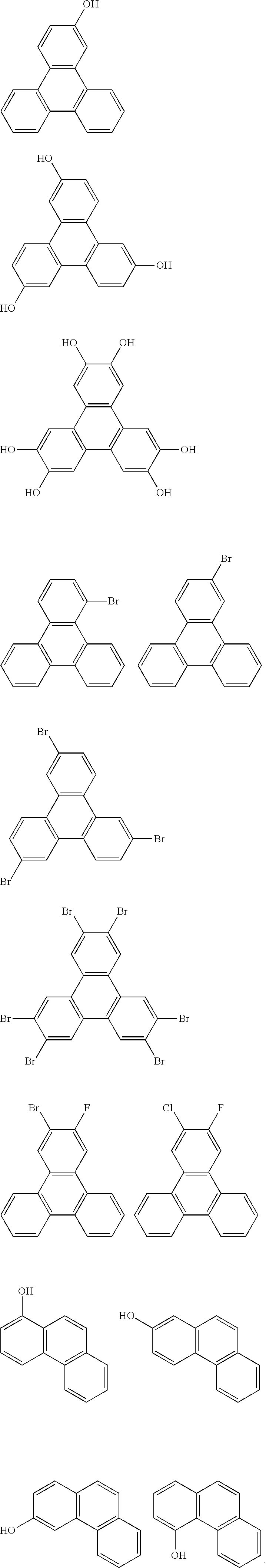

Theoretically, when all of the hydrogens in the deuterated compound are replaced with deuterium, that is, when the deuterium substitution rate is 100%, the service life characteristics are most ideally improved. However, there are problems such as the need for extreme conditions due to steric hindrance and the destruction of compound before the compound is deuterated due to side reactions, and in reality, it is difficult to obtain all the hydrogen of a compound at a deuterated substitution rate of 100%, and even when a deuterated substitution rate of nearly 100% is obtained, the efficiency compared to investment is not good in consideration of process time, cost, and the like.

In the present specification, since a deuterated compound produced by a deuterated reaction and having one or more deuteriums is produced as a composition having two or more isotopes having different molecular weights depending on the number of substituted deuteriums, the position where deuterium is substituted in the structure will be omitted.

In the compound having the structure, at least one of the positions which are indicated by hydrogen or in which substituted hydrogen is omitted can be substituted with deuterium.

The present specification provides an electronic device including the above-described deuterated aromatic compound.

The present specification provides a method for manufacturing an electronic device, the method including: manufacturing an electronic device using the above-described deuterated aromatic compound.

For the electronic device and the method for manufacturing an electronic device, the description of the composition above can be cited, and the repeated description will be omitted.

The electronic device is not particularly limited as long as the electronic device can use the above-described deuterated aromatic compound, and can be, for example, an organic light emitting device, an organic phosphorescent device, an organic solar cell, an organic photo conductor, an organic transistor, or the like.

The electronic device includes: a first electrode; a second electrode provided to face the first electrode; and an organic material layer having one or more layers provided between the first electrode and the second electrode, and one or more layers of the organic material layer can include the above-described deuterated aromatic compound.

The present specification provides an organic light emitting device including the above-described deuterated aromatic compound.

In an exemplary embodiment of the present specification, the organic light emitting device includes: a first electrode; a second electrode provided to face the first electrode; and an organic material layer provided between the first electrode and the second electrode, in which the organic material layer includes the deuterated aromatic compound.

In an exemplary embodiment of the present specification, the organic material layer includes a light emitting layer including the deuterated aromatic compound.

The organic material layer of the organic light emitting device of the present specification can also be composed of a single-layered structure, but can be composed of a multi-layered structure in which two or more organic material layers are stacked. For example, the organic material layer of the present specification can be composed of one to three layers. Further, the organic light emitting device of the present specification can have a structure including a hole injection layer, a hole transport layer, a light emitting layer, an electron transport layer, an electron injection layer, and the like as organic material layers. However, the structure of the organic light emitting device is not limited thereto, and can include a fewer number of organic layers.

When the organic light emitting device includes a plurality of organic material layers, the organic material layers can be formed of the same material or different materials.

For example, the organic light emitting device of the present specification can be manufactured by sequentially stacking a positive electrode, an organic material layer, and a negative electrode on a substrate. In this case, the organic light emitting device can be manufactured by depositing a metal or a metal oxide having conductivity, or an alloy thereof on a substrate to form a positive electrode, forming an organic material layer including a hole injection layer, a hole transport layer, a light emitting layer, and an electron transport layer thereon, and then depositing a material, which can be used as a negative electrode, thereon, by using a physical vapor deposition (PVD) method such as sputtering or e-beam evaporation. In addition to the method described above, an organic light emitting device can be made by sequentially depositing a negative electrode material, an organic material layer, and a positive electrode material on a substrate.

Further, the deuterated aromatic compound described above can be formed as an organic material layer by not only a vacuum deposition method, but also a solution application method when an organic light emitting device is manufactured. Here, the solution application method means spin coating, dip coating, doctor blading, inkjet printing, screen printing, a spray method, roll coating, and the like, but is not limited thereto.

In an exemplary embodiment of the present specification, the first electrode is a positive electrode, and the second electrode is a negative electrode.

According to another exemplary embodiment, the first electrode is a negative electrode, and the second electrode is a positive electrode.

In another exemplary embodiment, the organic light emitting device can be a normal type organic light emitting device in which a positive electrode, an organic material layer having one or more layers, and a negative electrode are sequentially stacked on a substrate.

In still another exemplary embodiment, the organic light emitting device can be an inverted type organic light emitting device in which a negative electrode, an organic material layer having one or more layers, and a positive electrode are sequentially stacked on a substrate.

In the present specification, the materials for the negative electrode, the organic material layer and the positive electrode are not particularly limited except for including an aromatic compound deuterated in at least one layer of the organic material layer, and a material known in the art can be used.

In the present specification, the above-described deuterated aromatic compound can be used by a principle which is similar to the principle applied to an organic light emitting device, even in an electronic device including an organic phosphorescent device, an organic solar cell, an organic photo conductor, an organic transistor, and the like. For example, the organic solar cell can have a structure including a negative electrode, a positive electrode, and a photoactive layer provided between the negative electrode and the positive electrode, and the photoactive layer can include the selected deuterated compound.

### EXAMPLES

Hereinafter, the present specification will be described in more detail through Examples. However, the following Examples are provided only for exemplifying the present specification, but are not intended to limit the present specification.

### EXAMPLES

### Example 1

35 ml of heavy water (D₂O) and 30 ml of cyclohexane were put into a flask, and 15 g of methanesulfonic anhydride and 5.0 g of 9-(naphthalene-1-yl)anthracene were slowly added dropwise thereto. Then, the reactants were allowed to react at 80° C. for 18 hours. After completion of the reaction, the temperature was lowered to room temperature (25° C.), and then ethyl acetate was added thereto. Then, the mixture was neutralized by adding potassium carbonate thereto, such that the pH became 7 to 8. After only the organic layer was isolated and the residual moisture was removed over magnesium sulfate (MgSO₄) therefrom, the residue was filtered and 9-(naphthalene-1-yl)anthracene substituted with deuterium was obtained by removing the solvent using a rotary evaporator.

### Example 2

9-(naphthalene-1-yl)anthracene substituted with deuterium was obtained by changing the organic solvent into methylcyclohexane instead of cyclohexane using the same method as in Example 1.

### Example 3

9-(naphthalene-1-yl)anthracene substituted with deuterium was obtained by changing the organic solvent into 1,4-dioxane instead of cyclohexane using the same method as in Example 1.

### Example 4

9-(naphthalene-1-yl)anthracene substituted with deuterium was obtained by changing the organic solvent into 1,2-dimethoxyethane instead of cyclohexane using the same method as in Example 1.

### Example 5

9-(naphthalene-1-yl)anthracene substituted with deuterium was obtained by changing the organic solvent into decalin instead of cyclohexane using the same method as in Example 1.

### Example 61

35 ml of heavy water (D₂O) and 30 ml of toluene were put into a flask, and 15 g of methanesulfonic anhydride and 5.0 g of 9-phenylanthracene were slowly put into the flask. Then, the reactants were allowed to react at 80° C. for 18 hours. After completion of the reaction, the temperature was lowered to room temperature (25° C.), and then ethyl acetate was added thereto. Then, the mixture was neutralized by adding potassium carbonate thereto, such that the pH became 7 to 8. After only the organic layer was isolated and the residual moisture was removed over magnesium sulfate (MgSO₄) therefrom, the residue was filtered and 9-phenylanthracene substituted with deuterium was obtained by removing the solvent using a rotary evaporator.

### Example 7

9-phenylanthracene substituted with deuterium was obtained by changing the organic solvent into xylene instead of toluene using the same method as in Example 6.

### Example 8

9-phenylanthracene substituted with deuterium was obtained by changing the organic solvent into chlorobenzene instead of toluene using the same method as in Example 6.

### Example 91

9-phenylanthracene substituted with deuterium was obtained by changing the organic solvent into 1,2-dichlorobenzene instead of toluene using the same method as in Example 6.

### Example 10

9-phenylanthracene substituted with deuterium was obtained by changing the organic solvent into 1,2,4-trichlorobenzene instead of toluene using the same method as in Example 6.

### Example 11

35 ml of heavy water (D₂O) and 30 ml of 1,1,1-trichloroethane were put into a flask, and 15 g of methanesulfonic anhydride and 5.0 g of 9-([1,1′-biphenyl]-4-yl)anthracene were slowly added dropwise thereto. Then, the reactants were allowed to react at 80° C. for 18 hours. After completion of the reaction, the temperature was lowered to room temperature (25° C.), and then ethyl acetate was added thereto. Then, the mixture was neutralized by adding potassium carbonate thereto, such that the pH became 7 to 8. After only the organic layer was isolated and the residual moisture was removed over magnesium sulfate (MgSO₄) therefrom, the residue was filtered and 9-([1,1′-biphenyl]-4-yl)-anthracene substituted with deuterium was obtained by removing the solvent using a rotary evaporator.

### Example 12

9-([1,1′-biphenyl]-4-yl)anthracene substituted with deuterium was obtained by changing the organic solvent into 1,1,2,2-tetrachloroethane instead of 1,1,1-trichloroethane using the same method as in Example 11.

### Example 13

9-([1,1′-biphenyl]-4-yl)anthracene substituted with deuterium was obtained by changing the organic solvent into 1,1,2,2-tetrachloroethylene instead of 1,1,1-trichloroethane using the same method as in Example 11.

### Example 14

9-(naphthalene-1-yl)anthracene substituted with deuterium was obtained by changing the anhydride into trifluoroacetic anhydride instead of methanesulfonic anhydride using the same method as in Example 1.

### Example 15

9-(naphthalene-1-yl)anthracene substituted with deuterium was obtained by changing the anhydride into acetic anhydride instead of methanesulfonic anhydride using the same method as in Example 1.

### Example 16

9-(naphthalene-1-yl)anthracene substituted with deuterium was obtained by changing the anhydride into trifluoromethanesulfonic anhydride instead of methanesulfonic anhydride using the same method as in Example 1.

### Comparative Example 1

In the present comparative example, a non-deuterated compound BH-A was used.

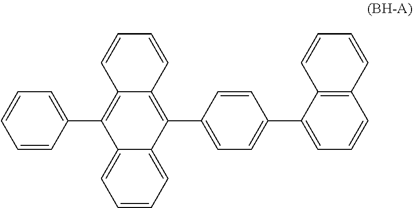 [SMILES: c1ccc(-c2c3ccccc3c(-c3ccc(-c4cccc5ccccc45)cc3)c3ccccc23)cc1]

### Example 17

30 ml of heavy water (D₂O) and 30 ml of cyclohexane were put into a flask, and 50 g of methanesulfonic anhydride and 5.0 g of Compound BH-A were slowly added dropwise thereto. Then, the reactants were allowed to react at 80° C. for 18 hours. After completion of the reaction, the temperature was lowered to room temperature (25° C.), and then ethyl acetate was added thereto. Then, the mixture was neutralized by adding potassium carbonate thereto, such that the pH became 7 to 8. After only the organic layer was isolated and the residual moisture was removed over magnesium sulfate (MgSO₄) therefrom, the residue was filtered and a compound BH-A substituted with deuterium was obtained by removing the solvent using a rotary evaporator.

### Example 18

9-bromoanthracene substituted with deuterium was obtained by changing the reactant into 9-bromoanthracene instead of 9-(naphthalene-1-yl)anthracene using the same method as in Example 1.

### Comparative Example 2

1 g of 9-(naphthalene-1-yl)anthracene, 15 ml of heavy water (D₂O), 0.5 g of 10% Pt/C, and 10 ml of a toluene solvent were put into a high-pressure reactor and the inside of the reactor was sealed by covering the head of the reactor. A gas including hydrogen was blown into the reactant for 3 to 5 minutes per minute with stirring. And then, the atmosphere in the reactor was maintained with the atmosphere of a gas, and the reaction was performed at a temperature of 130° C. for 24 hours. After completion of the reaction, the temperature was lowered, filtration was performed to remove the catalyst, and then heavy water was removed using MgSO₄, and then 9-(naphthalene-1-yl)anthracene substituted with deuterium was obtained by removing the solvent using a rotary evaporator.

### Comparative Example 3

A deuterium substitution reaction was performed by adding 9-bromoanthracene thereto instead of 9-(naphthalene-1-yl)anthracene using the same method as in Comparative Example 2. As a result, 9-bromoanthracene substituted with deuterium was obtained, but anthracene substituted with deuterium, which lost most of the bromine group could be confirmed.

### Experimental Example 1

The purity, deuterium substitution rate, and hydrogenated compound proportion for Examples 1 to 18 and Comparative Examples 2 to 3 were measured, and the results are shown in the following Table 1.

The purity and hydrogenated compound proportion were obtained by dissolving the completely reacted specimen in a tetrahydrofuran solvent for HPLC to integrate the spectrum at a wavelength of 254 nm through HPLC. In this case, as a mobile phase solvent, a solvent in which acetonitrile and tetrahydrofuran were mixed at a ratio of 5:5 and 1% formic acid was mixed and water were used.

A sample specimen obtained by quantifying a specimen completely subjected to deuterated reaction and dissolving the specimen in a solvent for NMR measurement, and an internal standard specimen obtained by quantifying any compound whose peak does not overlap with the compound before the deuterated reaction in the same amount as the above specimen and dissolving the compound in the same solvent for NMR measurement were prepared. NMR measurement graphs were obtained each using 1H-NMR for the prepared sample specimen and internal standard specimen.

When the 1H-NMR peak was assigned, a relative integration value for each position of the specimen completely subjected to deuterated reaction was obtained by setting the internal standard peak to 1.

When the specimen completely subjected to deuterated reaction is substituted with deuterium at all positions, no peak related to hydrogen appears, and in this case, the deuterium substitution rate is determined to be 100%. In contrast, when hydrogen at all positions is not substituted with deuterium, a peak of hydrogen that has not been substituted with deuterium will appear.

Based on this result, in the present experiment, a deuterium substitution rate is obtained by subtracting an integration value of a peak due to unsubstituted hydrogen in the NMR measurement graph of the sample specimen from an integration value of a peak related to hydrogen in the NMR measurement graph of the internal standard specimen in which deuterium is not substituted. This value is an integration value relative to each position, does not appear as the corresponding peak due to substitution with deuterium, and indicates a ratio of substitution with deuterium.

And then, a substitution rate for each position of the specimen was calculated using the weight of the specimen used when the 1H-NMR measurement sample is prepared, the weight of the internal standard, and the relative integration value.

TABLE 1

| | | | | Deuterium | Reaction | Reaction |
|---|---|---|---|---|---|---|
| | | | Purity | substitution rate | temperature | pressure |
| | Reactant | Organic solvent/Anhydride | (%) | (%) | (° C.) | (bar) |
| Example 1 | 9-(naphthalene-1-yl)- | Cyclohexane/Methanesulfonic anhydride | 98.2 | 60 | 80 | Normal |
| | anthracene | | | | | pressure |
| Example 2 | 9-(naphthalene-1-yl)- | Methylcyclohexane/Methanesulfonic anhydride | 98.7 | 66 | 100 | Normal |
| | anthracene | | | | | pressure |
| Example 3 | 9-(naphthalene-1-yl)- | 1,4-Dioxane/Methanesulfonic anhydride | 94.1 | 63 | 100 | Normal |
| | anthracene | | | | | pressure |
| Example 4 | 9-(naphthalene-1-yl)- | 1,2-Dimethoxyethane/Methanesulfonic | 94.5 | 65 | 100 | Normal |
| | anthracene | anhydride | | | | pressure |
| Example 5 | 9-(naphthalene-1-yl)- | Decalin/Methanesulfonic anhydride | 96.9 | 73 | 130 | Normal |
| | anthracene | | | | | pressure |
| Example 6 | 9-phenylanthracene | Toluene/Methanesulfonic anhydride | 97.0 | 69 | 110 | Normal |
| | | | | | | pressure |
| Example 7 | 9-phenylanthracene | Xylene/Methanesulfonic anhydride | 97.2 | 71 | 130 | Normal |
| | | | | | | pressure |
| Example 8 | 9-phenylanthracene | Chlorobenzene/Methanesulfonic anhydride | 97.4 | 74 | 120 | Normal |
| | | | | | | pressure |
| Example 9 | 9-phenylanthracene | 1,2-Dichlorobenzene/Methanesulfonic | 97.8 | 73 | 120 | Normal |
| | | anhydride | | | | pressure |
| Example 10 | 9-phenylanthracene | 1,2,4-Trichlorobenzene/Methanesulfonic | 97.3 | 76 | 120 | Normal |
| | | anhydride | | | | pressure |
| Example 11 | 9-([1,1′-biphenyl]-4- | 1,1,1-Trichloroethane/Methanesulfonic | 98.9 | 75 | 120 | Normal |
| | yl)anthracene) | anhydride | | | | pressure |
| Example 12 | 9-([1,1′-biphenyl]-4- | 1,1,2,2,-Tetrachloroethane/Methanesulfonic | 98.7 | 74 | 130 | Normal |
| | yl)anthracene) | anhydride | | | | pressure |
| Example 13 | 9-([1,1′-biphenyl]-4- | 1,1,2,2,-Tetrachloroethylene/Methanesulfonic | 97.8 | 72 | 120 | Normal |
| | yl)anthracene) | anhydride | | | | pressure |
| Example 14 | 9-(naphthalene-1-yl)- | Cyclohexane/Trifluoroacetic anhydride | 98.1 | 63 | 80 | Normal |
| | anthracene | | | | | pressure |
| Example 15 | 9-(naphthalene-1-yl)- | Cyclohexane/Acetic anhydride | 98.3 | 61 | 80 | Normal |
| | anthracene | | | | | pressure |
| Example 16 | 9-(naphthalene-1-yl)- | Cyclohexane/Trifluoromethanesulfonic | 98.7 | 78 | 80 | Normal |
| | anthracene | anhydride | | | | pressure |
| Example 17 | BH-A | Cyclohexane/Methanesulfonic anhydride | 98.9 | 79 | 80 | Normal |
| | | | | | | pressure |
| Example 18 | 9-bromoanthrancene | Cyclohexane/Methanesulfonic anhydride | 96.4 | 74 | 80 | Normal |
| | | | | | | pressure |
| Comparative | 9-(naphthalene-1-yl)- | Toluene (Pt/C) | 88.4 | 92.6 | 130 | 6.1 |
| Example 2 | anthracene | | | | | |
| Comparative | 9-bromoanthrancene | Toluene (Pt/C) | 47.9 | 82.9 | 145 | 6.3 |
| Example 3 | | | | | | |

Examples 1 to 18 can be performed under normal pressure without an increase in pressure during the reaction because the reaction is performed under acidic conditions. In contrast, in Comparative Examples 2 and 3, deuterium substitution was performed in a high-pressure reactor using a catalyst, but a deuterium substitution reaction occurred only when the reaction was performed under a pressure of normal pressure or more, that is, at least 5 bar or more. And when the reaction is performed using the high-pressure reactor, a side reaction occurs in which the double bond of the aromatic ring is partially reduced, resulting in a decrease in purity. In addition, it is difficult to separate the side reactants thus formed, and the yield is significantly reduced even during separation.

Example 18 and Comparative Example 3 are experiments in which a reaction was performed when the target compound had a leaving group. Even though starting materials are different, all of the target compounds each include a leaving group (—Br). Comparative Example 3 is an experiment in which deuterium was substituted under high pressure using a catalyst. These experiments are experiments to confirm whether the leaving group after the deuterium substitution reaction, is well attached without being detached, and in Example 18, the leaving group was well attached even after the deuterium substitution reaction, but in Comparative Example 3, a peak due to anthracene from which a bromine group, which is a leaving group, was detached was confirmed by HPLC-mass analysis.

### Experimental Example 2

### Comparative Device Example 1

A glass substrate thinly coated with indium tin oxide (ITO) to have a thickness of 1,400 Å was put into distilled water in which a detergent was dissolved, and ultrasonically washed. In this case, a Decon™ CON705 product manufactured by the Fischer Co., was used as the detergent, and distilled water twice filtered using a 0.22-μm sterilizing filter manufactured by Millipore Co., was used as the distilled water. After the ITO was washed for 30 minutes, ultrasonic washing was repeated twice by using distilled water for 10 minutes. After washing with distilled water was completed, the substrate was ultrasonically cleaned with each solvent of isopropyl alcohol, acetone and methanol for 10 minutes, dried, and then transported to a plasma cleaner. Furthermore, the substrate was cleaned by using oxygen plasma for 5 minutes, and then was transported to a vacuum deposition machine.

The following compounds HT and PD were thermally vacuum-deposited to have a thickness of 100 Å at a weight ratio of 95:5 on the transparent ITO electrode, which was thus prepared, thereby forming a hole injection layer, and only the HT material was subsequently vacuum-deposited to have a thickness of 1100 Å, thereby forming a hole transport layer. The following compound EB was thermally vacuum-deposited as an electron blocking layer thereon to have a thickness of 50 Å. Subsequently, the following compounds BH-A and BD were vacuum-deposited as a light emitting layer at a weight ratio of 96:4 to have a thickness of 200 Å. Subsequently, the following compounds ET and Liq were thermally vacuum-deposited as an electron transport layer at a weight ratio of 1:1 to have a thickness of 360 Å, and subsequently, the following compound Liq was vacuum-deposited to have a thickness of 5 Å, thereby forming an electron injection layer. A negative electrode was formed by subsequently depositing magnesium and silver at a weight ratio of 10:1 to have a thickness of 220 Å and aluminum to have a thickness of 1,000 Å on the electron injection layer, thereby manufacturing an organic light emitting device.

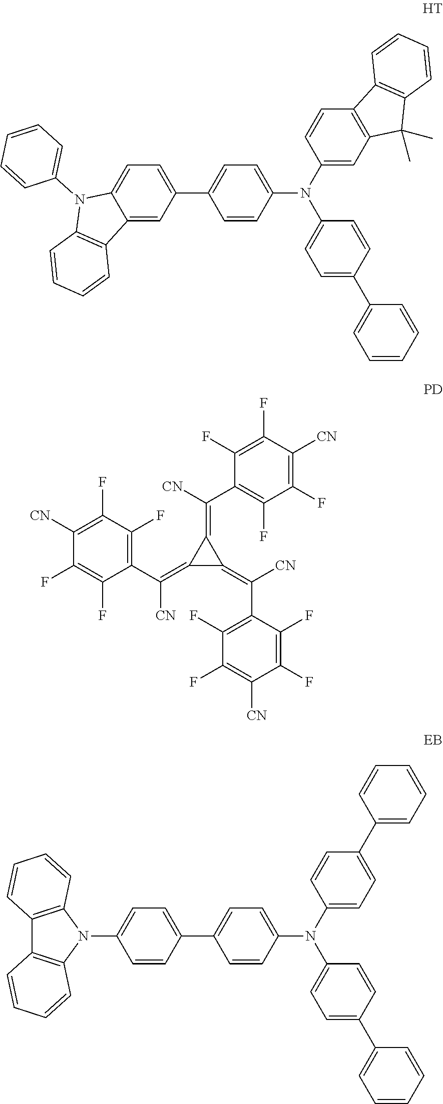

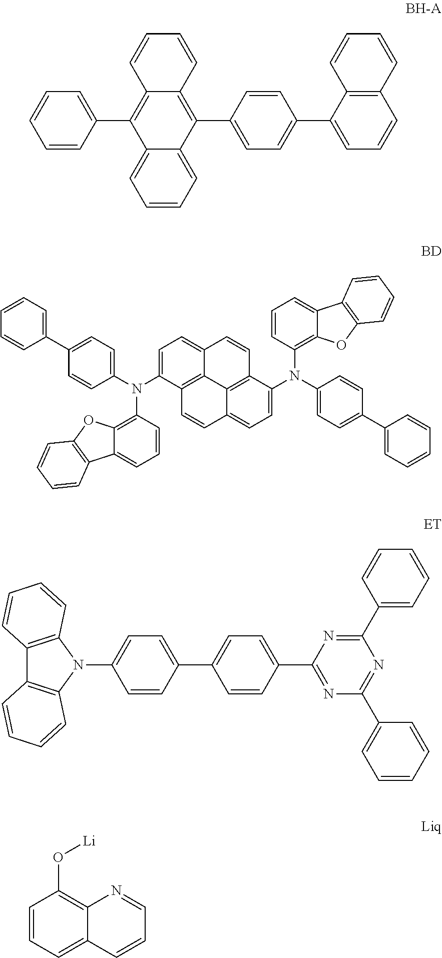

### Device Example 1

An organic light emitting device was manufactured in the same manner as in Comparative Device Example 1, except that the compounds (BH-A substituted with deuterium) prepared in Example 17 were used instead of BH-A as a host compound of the light emitting layer.

### Comparative Device Example 2 to Comparative Device Example 3

Organic light emitting devices were manufactured in the same manner as in Comparative Device Example 1, except that in Comparative Device Example 1, the compounds described in Table 2 were used instead of BH-A as a host compound of the light emitting layer. Further, the respective compounds of BH-B and BH-C in the following Table 2 are as follows.

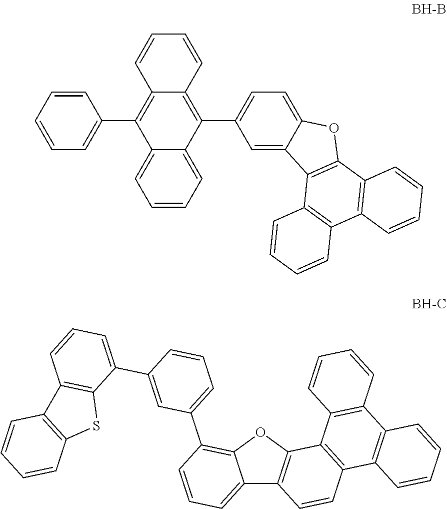 [SMILES: c1ccc(-c2c3ccccc3c(-c3ccc4oc5c6ccccc6c6ccccc6c5c4c3)c3ccccc23)cc1 ; c1cc(-c2cccc3c2oc2c3ccc3c4ccccc4c4ccccc4c32)cc(-c2cccc3c2sc2ccccc23)c1]

The voltage, efficiency, and service life (T95) were measured by applying a current to the organic light emitting devices manufactured in the previous Experimental Examples and Comparative Examples, and the results thereof are shown in the following Table 2. In this case, the voltage and efficiency were measured by applying a current density of 10 mA/cm², and T95 means the time taken for the initial luminance to be reduced to 95% at a current density of 20 mA/cm².

TABLE 2

| | | Voltage | Efficiency | Service life |
|---|---|---|---|---|
| | | (V) | (cd/A) | (T95, hr) |
| | Host | (@10 mA/ | (@10 mA/ | (@20 mA/ |
| | Material | cm²) | cm²) | cm²) |
| Device Example 1 | Example 17 | 4.64 | 5.86 | 80 |
| | (BH-A | | | |
| | substituted | | | |
| | with | | | |
| | deuterium) | | | |
| Comparative | BH-A | 4.95 | 5.33 | 45 |
| Device Example 1 | | | | |
| Comparative | BH-B | 4.72 | 5.10 | 25 |
| Device Example 2 | | | | |
| Comparative | BH-C | 9.55 | 0.21 | 5 |
| Device Example 3 | | | | |

1. A method for producing a deuterated aromatic compound, the method comprising: performing a deuterated reaction of an aromatic compound including one or more hydrocarbon aromatic rings using a solution comprising heavy water, an organic compound which can be hydrolyzed by the heavy water, the aromatic compound, and an organic solvent.

2. The method of claim 1, wherein the organic solvent is selected from the group consisting of a hydrocarbon chain which is unsubstituted or substituted with a group selected from an alkyl group and a halogen group; an aliphatic hydrocarbon ring which is unsubstituted or substituted with a group selected from an alkyl group and a halogen group; an aromatic hydrocarbon ring which is unsubstituted or substituted with a group selected from an alkyl group and a halogen group; an alkene compound which is unsubstituted or substituted with a group selected from an alkyl group and a halogen group; a straight-chained or branched heterochain; a substituted or unsubstituted aliphatic hetero ring; and a substituted or unsubstituted aromatic hetero ring.

3. The method of claim 1, wherein the organic solvent is selected from the group consisting of cyclohexane, methylcyclohexane, ethylcyclohexane, chlorocyclohexane, dioxane, tetrahydrofuran, diethyl ether, 1,2-dimethoxyethane, decalin, hexane, heptane, toluene, xylene, mesitylene, dichloromethane, 1,2-dichloroethane, 1,1,1-trichloroethane, 1,1,2,2-tetrachloroethane, 1,1,2,2-tetrachloroethylene, chlorobenzene, 1,2-dichlorobenzene and 1,2,4-trichlorobenzene.

4. The method of claim 1, wherein the organic compound which can be hydrolyzed by the heavy water comprises at least one compound of the following Chemical Formulae 1 to 4:
R1-C(O)OC(O)—R2 [Chemical Formula 1]
R3-S(O₂)OS(O₂)—R4 [Chemical Formula 2]
R5-C(O)O—R6 [Chemical Formula 3]
R7-CONH—R8 [Chemical Formula 3]
wherein in Chemical Formulae 1 to 4:
R1 to R8 are the same as or different from each other, and are each independently a monovalent organic group which is unsubstituted or substituted with a halogen group.

5. The method of claim 1, wherein the organic compound which can be hydrolyzed by the heavy water comprises at least one of trifluoromethanesulfonic anhydride, trifluoroacetic anhydride, acetic anhydride, methanesulfonic anhydride, methyl acetate, ethyl acetate and dimethylacetamide.

6. The method of claim 1, wherein the performing of the deuterated reaction of the aromatic compound comprises:
preparing a solution comprising an aromatic compound comprising one or more hydrocarbon aromatic rings, heavy water, an organic compound which can be hydrolyzed by the heavy water, and an organic solvent; and
performing the deuterated reaction of the aromatic compound by heating the solution.

7. The method of claim 6, wherein a temperature at which the deuterated reaction of the aromatic compound is performed by heating the solution is 80° C. or more and 140° C. or less.

8. A deuterated reaction composition comprising an aromatic compound comprising one or more hydrocarbon aromatic rings, heavy water, an organic compound which can be hydrolyzed by the heavy water, and an organic solvent.

9. The deuterated reaction composition of claim 8, wherein the organic solvent is selected from the group consisting of a hydrocarbon chain which is unsubstituted or substituted with a group selected from an alkyl group and a halogen group; an aliphatic hydrocarbon ring which is unsubstituted or substituted with a group selected from an alkyl group and a halogen group; an aromatic hydrocarbon ring which is unsubstituted or substituted with a group selected from an alkyl group and a halogen group; an alkene compound which is unsubstituted or substituted with a group selected from an alkyl group and a halogen group; a straight-chained or branched heterochain; a substituted or unsubstituted aliphatic hetero ring; and a substituted or unsubstituted aromatic hetero ring.

10. The deuterated reaction composition of claim 8, wherein the organic solvent is selected from the group consisting of cyclohexane, methylcyclohexane, ethylcyclohexane, chlorocyclohexane, dioxane, tetrahydrofuran, diethyl ether, 1,2-dimethoxyethane, decalin, hexane, heptane, toluene, xylene, mesitylene, dichloromethane, 1,2-dichloroethane, 1,1,1-trichloroethane, 1,1,2,2-tetrachloroethane, 1,1,2,2-tetrachloroethylene, chlorobenzene, 1,2-dichlorobenzene and 1,2,4-trichlorobenzene.

11. A deuterated aromatic compound produced by the method of claim 1 that is any one compound of the following Chemical Formulae 7 to 10: 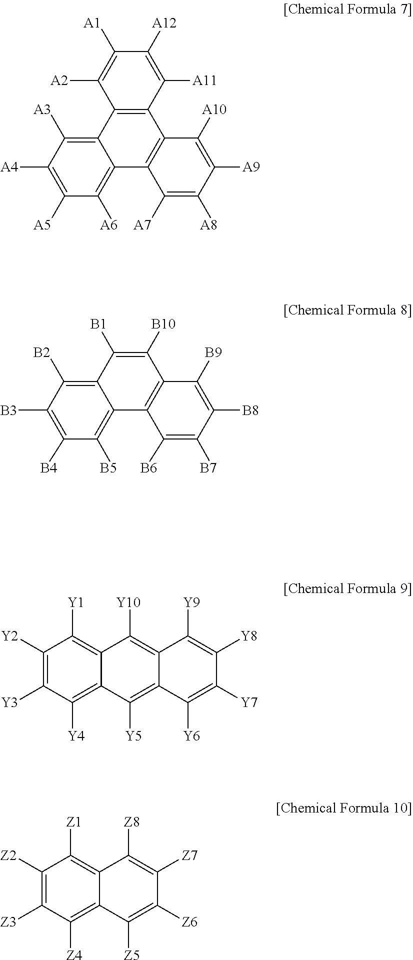
wherein in Chemical Formulae 7 to 10:
at least one of A1 to A12 is deuterium, at least one is a substituent selected from the group consisting of a leaving group, a hydroxyl group, a substituted or unsubstituted amine group and a cyano group, and the others are each independently hydrogen, a leaving group, a hydroxyl group, a substituted or unsubstituted amine group, a cyano group, a substituted or unsubstituted alkyl group, a substituted or unsubstituted cycloalkyl group, a substituted or unsubstituted aryl group, or a substituted or unsubstituted heterocyclic group, or are optionally bonded to an adjacent group to form a substituted or unsubstituted hydrocarbon ring;
at least one of B1 to B10 is deuterium, at least one is a substituent selected from the group consisting of a leaving group, a hydroxyl group, a substituted or unsubstituted amine group and a cyano group, and the others are each independently hydrogen, a leaving group, a hydroxyl group, a substituted or unsubstituted amine group, a cyano group, a substituted or unsubstituted alkyl group, a substituted or unsubstituted cycloalkyl group, a substituted or unsubstituted aryl group, or a substituted or unsubstituted heterocyclic group, or are optionally bonded to an adjacent group to form a substituted or unsubstituted hydrocarbon ring;
at least one of Y1 to Y10 is deuterium, at least one is a substituent selected from the group consisting of a leaving group, a hydroxyl group, a substituted or unsubstituted amine group and a cyano group, and the others are each independently hydrogen, a leaving group, a hydroxyl group, a substituted or unsubstituted amine group, a cyano group, a substituted or unsubstituted alkyl group, a substituted or unsubstituted cycloalkyl group, a substituted or unsubstituted aryl group, or a substituted or unsubstituted heterocyclic group, or are optionally bonded to an adjacent group to form a substituted or unsubstituted hydrocarbon ring; and
at least one of Z1 to Z8 is deuterium, at least one is a substituent selected from the group consisting of a leaving group, a hydroxyl group, a substituted or unsubstituted amine group and a cyano group, and the others are each independently hydrogen, a leaving group, a hydroxyl group, a substituted or unsubstituted amine group, a cyano group, a substituted or unsubstituted alkyl group, a substituted or unsubstituted cycloalkyl group, a substituted or unsubstituted aryl group, or a substituted or unsubstituted heterocyclic group, or are optionally bonded to an adjacent group to form a substituted or unsubstituted hydrocarbon ring.

12. (canceled)

13. The deuterated aromatic compound of claim 11, wherein the leaving group is selected from the group consisting of a halogen group and a boronic acid group.

14. (canceled)

15. The deuterated aromatic compound of claim 11, wherein the deuterated aromatic compound comprising the substituent selected from the group consisting of a leaving group, a hydroxyl group, a substituted or unsubstituted amine group and a cyano group is any one compound of the following compounds, and the compounds are each substituted with one or more deuteriums:

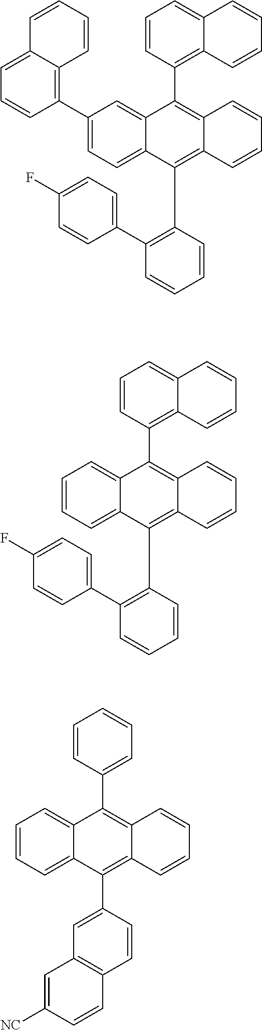 [SMILES: Fc1ccc(-c2ccccc2-c2c3ccccc3c(-c3cccc4ccccc34)c3cc(-c4cccc5ccccc45)ccc23)cc1 ; Fc1ccc(-c2ccccc2-c2c3ccccc3c(-c3cccc4ccccc34)c3ccccc23)cc1 ; N#Cc1ccc2ccc(-c3c4ccccc4c(-c4ccccc4)c4ccccc34)cc2c1]

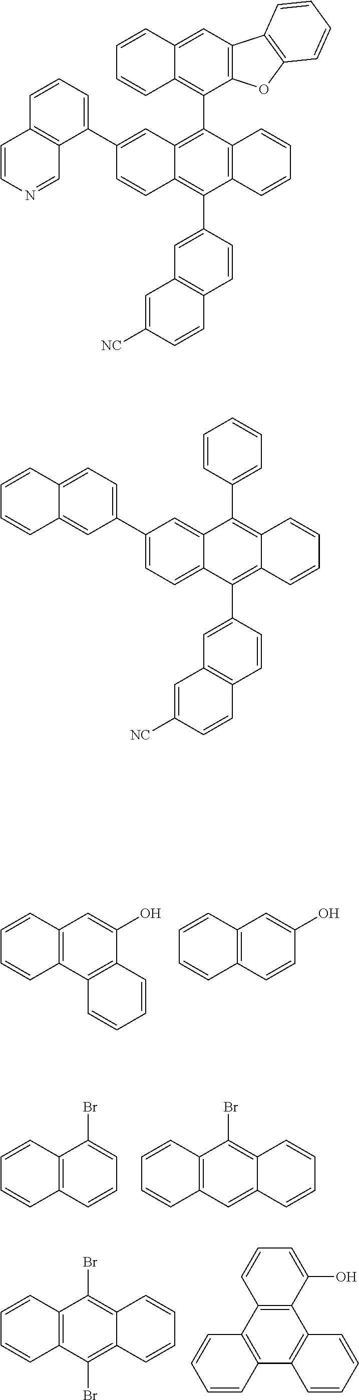

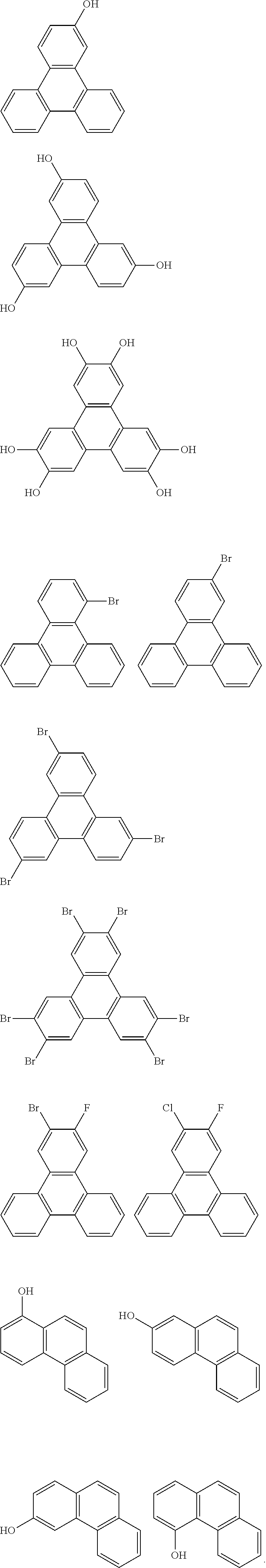

16. An electronic device comprising the deuterated aromatic compound of claim 11.
